(19) United States
(12) Patent Application Publication
(10) Pub. No.: US 2026/0308119 A1
(43) Pub. Date: Oct. 8, 2026

(54) MINING CHEMICAL COLLECTOR COMPOSITIONS AND PROCESSES FOR RECOVERING METAL FROM ORE

(71) Applicant: Chevron Phillips Chemical Company LP, The Woodlands, TX (US)

(72) Inventors: Christophe LAROCHE, Bartlesville, OK (US); R. Shawn CHILDRESS, Bartlesville, OK (US); Henry LOPEZ, Bartlesville, OK (US); Chad W. BROWN, Bartlesville, OK (US); Debbie G. LANEY, Elko, NV (US); Robert M. TRIEBEL, Dewey, OK (US)

(21) Appl. No.: 19/577,121

(22) Filed: Mar. 24, 2026

Related U.S. Application Data
(60) Provisional application No. 63/779,936, filed on Mar. 28, 2025.

(51) Int. Cl. C07C 327/08; B03D 1/02

(52) U.S. Cl. CPC C07C 327/08; B03D 1/02

(57) ABSTRACT

Aspects of the present disclosure generally relate to a new class of mining chemical collector compositions and uses thereof. Disclosed herein is a mining chemical collector composition that includes a first collector agent comprising one or more alkylthiocarboxylic acids. Also disclosed herein is a process for recovery of a metal from an ore. The process includes contacting an ore with a mining chemical collector composition that includes one or more alkylthiocarboxylic acids.

### CROSS-REFERENCE TO RELATED APPLICATIONS

This application claims benefit of and priority to U.S. Provisional Patent Application Ser. No. 63/779,936, filed on Mar. 28, 2025, which is incorporated herein by reference in its entirety.

### FIELD

Aspects of the present disclosure generally relate to a new class of mining chemical collector compositions. Aspects of the present disclosure also generally relate to uses of such compositions, such as in processes for recovering a metal from an ore.

### BACKGROUND

Mining chemical collectors are utilized during mineral recovery processes such as froth flotation. Froth flotation is a process for separating and concentrating desired, valuable minerals, such as metal sulfides and metal oxides, from undesired gangue minerals. The separation relies heavily on hydrophobicity differences between the desired minerals and the gangue, and such hydrophobicity differences are enhanced by the use of a chemical collector. Therefore, the efficiency and success of froth flotation relies on the ability of a chemical collector to selectively recover different minerals into their respective concentrates at good recoveries and grades.

Many ores contain mixtures of oxide and sulfide minerals. However, such ores are beneficiated using conventional chemical collectors to recover either oxide ore particles or sulfide ore particles, not both. Therefore, conventional chemical collectors are ineffective at recovering mixed sulfide/oxide ore particles.

There is a need for new mining chemical collector compositions and uses thereof.

### SUMMARY

Aspects of the present disclosure generally relate to a new class of mining chemical collector compositions and uses thereof, such as in processes for recovering a metal from an ore. The new mining chemical collector compositions include, for example, an alkylthiocarboxylic acid. Alkylthiocarboxylic acids include two useful functional groups: a sulfide ether linkage and a carboxylic acid. The sulfide ether linkage of the alkylthiocarboxylic acid has the ability to interact with and/or attach to the sulfide part of a mixed sulfide/oxide ore particle. The carboxylic acid of the alkylthiocarboxylic acid has the ability to interact with and/or attach to the oxide part of the mixed sulfide/oxide ore particle. As a result, and advantageously, the presence of the two functional groups in a single chemical collector enables efficient attachment of the alkylthiocarboxylic acid to the surface of the mixed sulfide/oxide ore particle, increasing its hydrophobicity. The increased hydrophobicity of the mixed sulfide/oxide ore particle is more amenable to being recovered using froth flotation techniques, enabling, for example, improved percent metal recovery, improved grade of metal recovered, or combinations thereof relative to conventional technologies.

In an aspect, a mining chemical collector composition is provided. The mining chemical collector composition includes a first collector agent comprising one or more alkylthiocarboxylic acids, each alkylthiocarboxylic acid represented by formula (I):

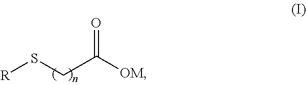

wherein: R¹ of formula (I) is a C4-C20 hydrocarbyl; n of formula (I) is 1 or 2; and M of formula (I) is hydrogen (H), Li, Na, K, Rb, or Cs.

In another aspect, a copper collector composition is provided. The copper collector composition includes a mining chemical collector composition described herein.

In another aspect, a molybdenum collector composition is provided. The molybdenum collector composition includes a mining chemical collector composition described herein.

In another aspect, a flotation composition is provided. The flotation composition includes a mining chemical collector composition described herein.

In another aspect, a process for recovery of a metal from an ore is provided. The process includes contacting an ore with a mining chemical collector composition described herein.

In another aspect is provided a composition that includes an ore and a mining chemical collector composition described herein.

### BRIEF DESCRIPTION OF THE DRAWINGS

So that the manner in which the above recited features of the present disclosure can be understood in detail, a more particular description of the disclosure, briefly summarized above, can be had by reference to aspects, some of which are illustrated in the appended drawings. It is to be noted, however, that the appended drawings illustrate only exemplary aspects and are therefore not to be considered limiting of its scope, can admit to other equally effective aspects.

FIG. 1A shows selected data for the recovery of copper from an ore (Mine A) utilizing examples of mining chemical collector compositions described herein and comparative examples.

FIG. 1B shows selected data for the recovery of iron from an ore (Mine A) utilizing examples of mining chemical collector compositions described herein and comparative examples.

FIG. 1C shows selected data for the recovery of molybdenum from an ore (Mine A) utilizing examples of mining chemical collector compositions described herein and comparative examples.

FIG. 2A shows selected data for the recovery of copper from an ore (Mine B) utilizing an example mining chemical collector composition described herein and comparative examples.

FIG. 2B shows selected data for the recovery of iron from an ore (Mine B) utilizing an example mining chemical collector composition described herein and comparative examples.

FIG. 2C shows selected data for the recovery of molybdenum from an ore (Mine B) utilizing an example mining chemical collector composition described herein and comparative examples.

To facilitate understanding, identical reference numerals have been used, where possible, to designate identical elements that are common to the figures. It is contemplated that elements and features of one aspect can be beneficially incorporated in other aspects without further recitation.

### DETAILED DESCRIPTION

Aspects of the present disclosure generally relate to a new class of mining chemical collector compositions and uses thereof, such as in processes for recovering a metal from an ore. As described herein, mining chemical collector compositions can be used for the beneficiation of ores, such as during a froth flotation process.

Froth flotation processes enable the separation and concentration of target minerals from undesired gangue minerals and generally include the following operations: Ore is crushed or ground to small particles. The small particles of ore are diluted with water to a mineral pulp having a certain % solids by weight. Collectors and other agents are added to the pulp and the pulp is placed in a flotation cell. The pulp is aerated, producing air bubbles. The hydrophobic air bubbles selectively attach to those particles rendered hydrophobic by action of the collectors utilized. The hydrophobic particles and hydrophobic air bubbles rise to the surface forming a froth, and the hydrophilic particles, referred to as tailings, remain in the flotation cell. The froth is then skimmed from the flotation cell, thereby producing a concentrate of a target mineral. In direct flotation systems, the collector renders hydrophobicity to the desired mineral particles. In reverse flotation systems, the collector renders hydrophobicity to the waste mineral particles. Overall, the collector plays a large role in the success of a froth flotation process.

As described above, conventional chemical collectors are ineffective at recovering mixed sulfide/oxide ore particles. Therefore, there is a need for new mining chemical collector compositions and uses thereof. To this end, the inventors discovered a new class of mining chemical collector compositions that include an alkylthiocarboxylic acid. In contrast to conventional chemical collectors, alkylthiocarboxylic acids described herein are able to effectively recover mixed sulfide/oxide ore particles and provide a beneficial balance of, for example, percent metal recovery, grade of metal recovered, metal selectivity, and percent mass recovered.

The alkylthiocarboxylic acid can be used with a mercaptan. In such cases, the inventors discovered a synergistic effect between the alkylthiocarboxylic acid and the optional mercaptan. The synergy between these components can result in, for example, improved percent metal recovery, improved grade of metal recovered, improved metal selectivity, or combinations thereof relative to conventional technologies. The synergy can result in a lower percent mass recovered from the ore (e.g., a lower total mass recovered from the ore) relative to conventional technologies. A lower percent mass recovered can be beneficial for further processing as lower percent masses recovered can be cheaper and more efficient for downstream processing relative to higher percent masses recovered (higher total masses recovered). Additionally, aspects described herein can provide for improved selectivity against iron (for example, iron-containing minerals and compounds) relative to conventional approaches.

Aspects of the present disclosure generally relate to a new class of mining chemical collector compositions. A “composition” can include component(s) of the composition, reaction product(s) of two or more components of the composition, a remainder balance of remaining starting component(s), or combinations thereof. The mining chemical collector compositions can be utilized for the beneficiation of ores, for example, during any suitable froth flotation process. Compositions described herein can be useful in any suitable process to separate metals and metal-containing materials.

Mining chemical collector compositions described herein can include a first collector agent. The first collector agent can include one or more alkylthiocarboxylic acids. Each of the one or more alkylthiocarboxylic acids can be represented by formula (I):

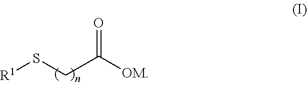

In formula (I), n can be any suitable integer, for example, 1, 2, or 3, such as 1 or 2. In formula (I), M can be hydrogen (H), or a cation such as a monovalent cation such as Li, Na, K, Rb, or Cs. When M of formula (I) is a monovalent cation, the alkylthiocarboxylic acid represented by formula (I) is a salt.

R¹ of formula (I) can be any suitable hydrocarbyl group, such as an unsubstituted hydrocarbyl or a substituted hydrocarbyl.

The term “hydrocarbyl group” is used herein in accordance with the definition specified by IUPAC: a univalent group formed by removing a hydrogen atom from a hydrocarbon. “Hydrocarbon” refers to a compound containing only carbon and hydrogen. Hydrocarbyl groups can be linear or branched, saturated or unsaturated, cyclic or acyclic, aromatic or non-aromatic. Non-limiting examples of hydrocarbyl groups can include alkyl, alkenyl, cycloalkyl, aryl, and aralkyl groups, amongst other groups, such as ethyl, phenyl, tolyl, propenyl, and the like.

An “unsubstituted hydrocarbyl” refers to a group that consists of hydrogen and carbon atoms only. Non-limiting examples of unsubstituted hydrocarbyl include an alkyl group having from 1 to 30 carbon atoms such as methyl, ethyl, n-propyl, isopropyl, n-butyl, iso-butyl, sec-butyl, and tert-butyl, pentyl, hexyl, heptyl, octyl, ethyl-2-hexyl, isooctyl, nonyl, n-decyl, isodecyl, or isomers thereof, a cycloaliphatic group having from 3 to 30 carbon atoms such as, for example, cyclopentyl or cyclohexyl; an aromatic group having from 6 to 30 carbon atoms such as, for example, phenyl, naphthyl, or fluorenyl; or any combination thereof. A “substituted hydrocarbyl” refers to an unsubstituted hydrocarbyl in which at least one hydrogen of the unsubstituted hydrocarbyl has been substituted with at least one heteroatom or heteroatom-containing group, such as one or more elements from Group 13-17 of the periodic table of the elements, such as halogen (F, Cl, Br, or I), O, N, Se, Te, P, As, Sb, S, B, Si, Ge, Sn, Pb, and the like, such as C(O)R*, C(C)NR*₂, C(O)OR*, NR*₂, OR*, SeR*, TeR*, PR*₂, AsR*₂, SbR*₂, SR*, SOₓ (where x=2 or 3), BR*₂, SiR*₃, GeR*₃, SnR*₃, PbR*₃, and the like, where R* is, independently, hydrogen or unsubstituted hydrocarbyl, or where at least one heteroatom has been inserted within the unsubstituted hydrocarbyl. Substituted includes inorganic and organic substituents.

Referring back to formula (I), R¹ of formula (I) can be linear or branched, saturated or unsaturated, cyclic or acyclic, aromatic or non-aromatic. Regarding saturation, R¹ of formula (I) can be fully saturated, partially unsaturated, or fully unsaturated. R¹ of formula (I) can have any suitable number of carbon atoms. For example, R¹ of formula (I) can be a C1-C30 hydrocarbyl, such as a C2-C28 hydrocarbyl, such as a C3-C26 hydrocarbyl, such as a C4-C24 hydrocarbyl, such as a C5-C22 hydrocarbyl, such as a C6-C20 hydrocarbyl, or a C4-C20 hydrocarbyl, such as a C5-C18 hydrocarbyl, such as a C6-C16 hydrocarbyl, such as a C7-C14 hydrocarbyl, such as a C7-C11 hydrocarbyl. R¹ of formula (I) can include at least one double bond, such as 1, 2, or 3 double bonds. A double bond is also referred to as an unsaturated bond. When more than one alkylthiocarboxylic acid represented by formula (I) is present in mining chemical collector compositions described herein, the one or more alkylthiocarboxylic acids represented by formula (I) are different.

Illustrative, but non-limiting, examples of R¹ of formula (I) can include n-hexyl (where R¹ of formula (I) has 6 carbons), cyclohexyl (where R¹ of formula (I) has 6 carbons), n-octyl (where R¹ of formula (I) has 8 carbons), n-decyl (where R¹ of formula (I) has 10 carbons), n-dodecyl (where R¹ of formula (I) has 12 carbons), a branched C10 hydrocarbyl (where R¹ of formula (I) has 10 carbons), or a branched C12 hydrocarbyl (where R¹ of formula (I) has 12 carbons).

When R¹ of formula (I) is a branched C10 hydrocarbyl, the alkylthiocarboxylic acid can be derived from reaction (such as a thiol-ene type reaction) of a branched monoolefin with a suitable sulfur-containing molecule such as thioglycolic acid, 3-mercaptopropionic acid, or other suitable sulfur-containing molecule. Such branched C10 monoolefins can include 5-methyl-1-nonene (represented by structure A), 3-propyl-1-heptene (represented by structure B), 4-ethyl-1-octene, (represented by structure C), 2-butyl-1-hexene (represented by structure D), or combinations thereof.

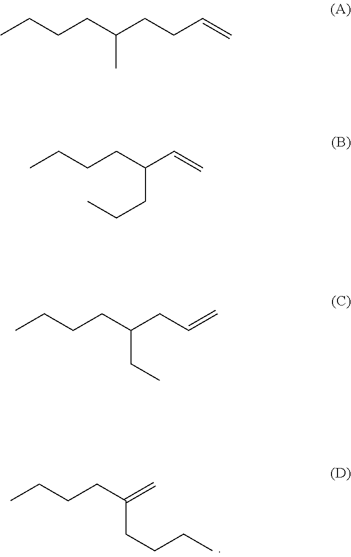

The one or more alkylthiocarboxylic acids represented by formula (I) can include one or more alkylthiocarboxylic acids represented by formula (I-A), formula (I-B), formula (I-C), formula (I-D), formula (I-E), formula (I-F), formula (I-G), formula (I-H), formula (I-I), formula (I-J), formula (I-K), formula (I-L), or combinations thereof.

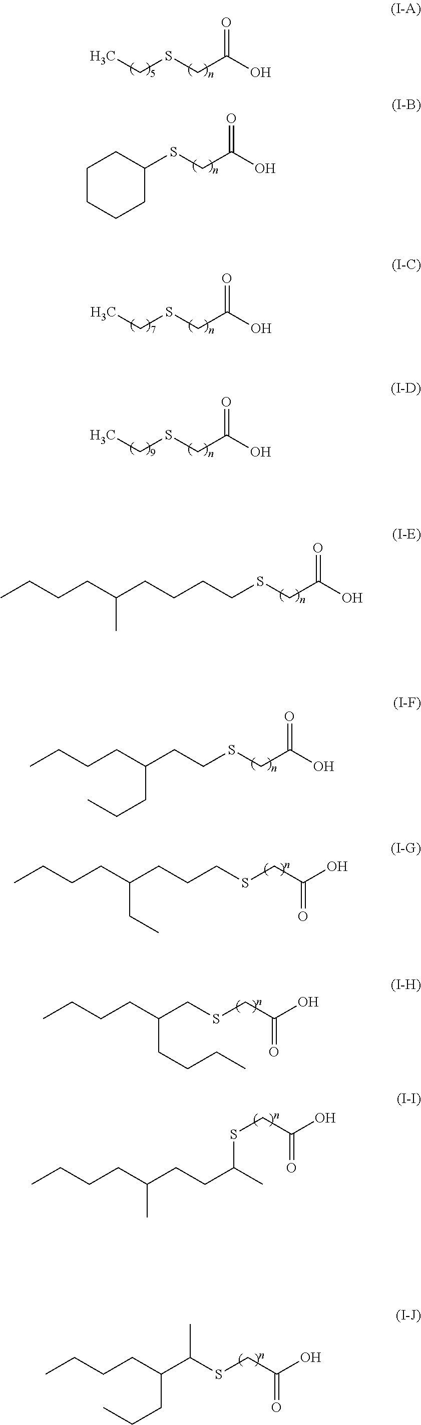

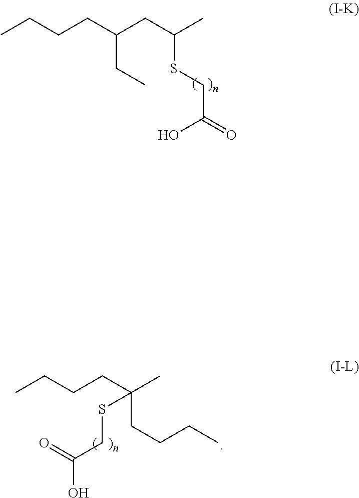

The “n” of formulas (I-A), (I-B), (I-C), (I-D), (I-E), (I-F), (I-G), (I-H), (I-I), (I-J), (I-K), and (I-L) can be, independently, 1 or 2.

The hydrogen of the carboxylic acid groups shown in formulas (I-A), (I-B), (I-C), (I-D), (I-E), (I-F), (I-G), (I-H), (I-I), (I-J), (I-K), and (I-L) can be replaced with a monovalent cation such as Li, Na, K, Rb, or Cs.

Illustrative, but non-limiting, examples of alkylthiocarboxylic acids can include structure (CL-1), structure (CL-2), structure (CL-3), structure (CL-4), structure (CL-5), structure (CL-6), or combinations thereof.

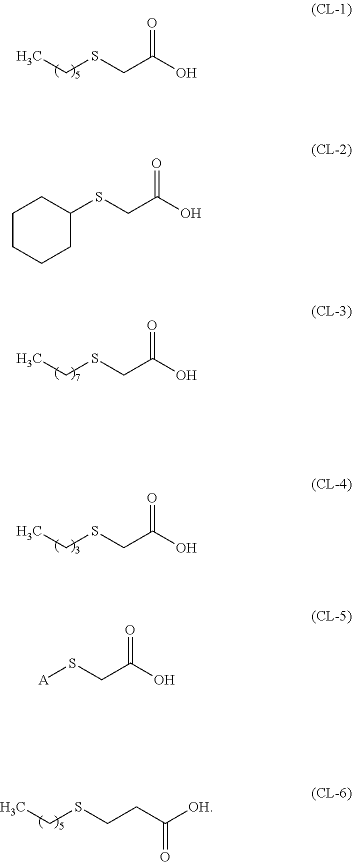

The hydrogen of the carboxylic acid groups shown in structure (CL-1), structure (CL-2), structure (CL-3), structure (CL-4), structure (CL-5), structure (CL-6) can be replaced with a monovalent cation such as Li, Na, K, Rb, or Cs.

For structure CL-5, “A” of structure CL-5 can be a branched C10 hydrocarbyl. In some aspects, which can be combined with other aspects, structure CL-5 can include one or more of the following compounds:

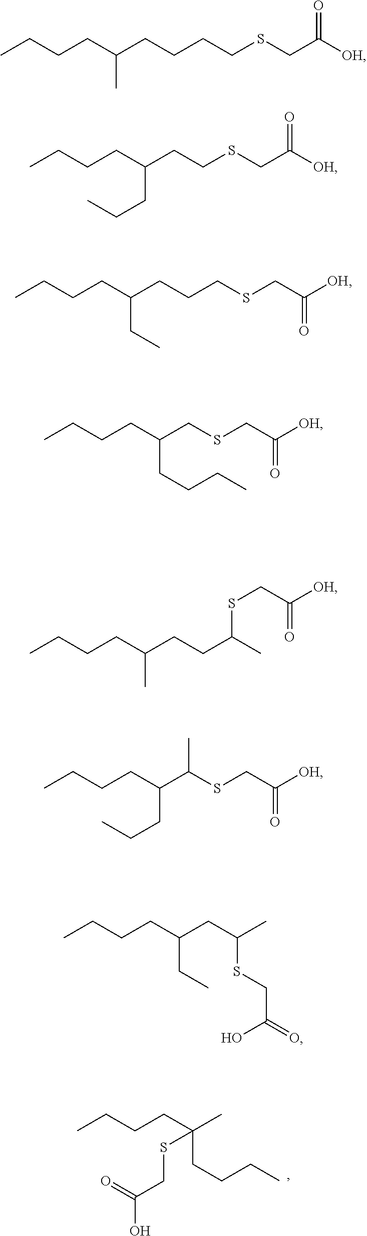

or combinations thereof.

The hydrogen of the carboxylic acid groups can be replaced with a monovalent cation such as Li, Na, K, Rb, or Cs.

Accordingly, structure CL-5 can be a mixture of compounds, wherein each R¹ of formula (I) of the mixture of compounds is a branched C10 hydrocarbyl.

As described herein, alkylthiocarboxylic acids include a sulfide ether linkage moiety and a carboxylic acid moiety. The sulfide ether linkage moiety can interact with and/or attach to a sulfide part of a mixed sulfide/oxide ore particle. Mixed sulfide/oxide ore particles are interchangeably referred to herein as mixed sulfide/oxide particles. The carboxylic acid moiety of the alkylthiocarboxylic acid can interact with and/or attach to an oxide part of the mixed sulfide/oxide particle. As a result, the presence of the two functional groups in a single chemical collector can enable efficient attachment of the alkylthiocarboxylic acid to the surface of the mixed sulfide/oxide particle and increase its hydrophobicity. In turn, the increased hydrophobicity of the mixed sulfide/oxide particle is more amenable to being recovered using froth flotation techniques, enabling, for example, improved percent metal recovery, improved grade of metal recovered, or combinations thereof, among other improvements relative to conventional technologies.

Mining chemical collector compositions described herein can optionally include a second collector agent. The optional second collector agent can optionally include one or more mercaptans. Mercaptans are also known as thiols. Each of the optional one or more mercaptans can be represented by formula (II):

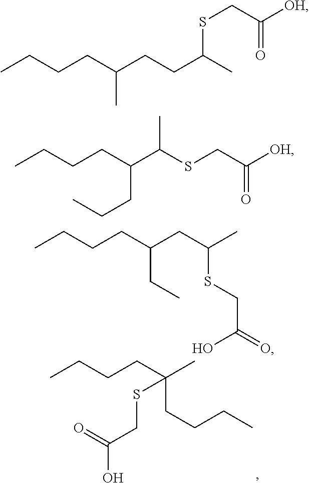

R² of formula (II) can be any suitable hydrocarbyl group, such as an unsubstituted hydrocarbyl or a substituted hydrocarbyl. R² of formula (II) can be linear or branched, saturated or unsaturated, cyclic or acyclic, aromatic or non-aromatic. Regarding saturation, R² of formula (II) can be fully saturated, partially unsaturated, or fully unsaturated.

R² of formula (II) can have any suitable number of carbon atoms. For example, R² of formula (II) can be a C1-C30 hydrocarbyl, such as a C2-C26 hydrocarbyl, such as a C3-C22 hydrocarbyl, such as a C4-C20 hydrocarbyl, such as a C5-C18 hydrocarbyl, such as a C6-C16 hydrocarbyl. R² of formula (II) can include at least one double bond, such as 1, 2, or 3 double bonds. When more than one optional mercaptan represented by formula (II) is present in mining chemical collector compositions described herein, the optional one or more mercaptans represented by formula (II) are different.

Illustrative, but non-limiting, examples of R² of formula (II) can include n-hexyl (where R² of formula (II) has 6 carbons), cyclohexyl (where R² of formula (II) has 6 carbons), n-octyl (where R² of formula (II) has 8 carbons), n-decyl (where R² of formula (II) has 10 carbons), n-dodecyl (where R² of formula (II) has 12 carbons), a branched C10 hydrocarbyl (where R² of formula (II) has 10 carbons), or a branched C12 hydrocarbyl (where R² of formula (II) has 12 carbons).

The optional one or more mercaptans represented by formula (II) can include n-decyl mercaptan, n-dodecyl mercaptan, tert-dodecyl mercaptan, or combinations thereof. The n-dodecyl mercaptan can include Orfom® CO100, commercially available from Chevron Phillips Chemical Company. The tert-dodecyl mercaptan can include Orfom® CO210, commercially available from Chevron Phillips Chemical Company.

The optional one or more mercaptans represented by formula (II) can include a branched C10 mercaptan. Illustrative, but non-limiting, examples of the branched C10 mercaptan can include: 5-methyl-1-mercapto-nonane (represented by Structure A), 3-propyl-1-mercapto-heptane (represented by structure B), 4-ethyl-1-mercapto-octane (represented by structure C), 2-butyl-1-mercapto-hexane (represented by structure D), 5-methyl-2-mercapto-nonane (represented by structure E), 3-propyl-2-mercapto-heptane (represented by structure F), 4-ethyl-2-mercapto-octane (represented by structure G), 5-methyl-5-mercapto-nonane (represented by structure H), or combinations thereof.

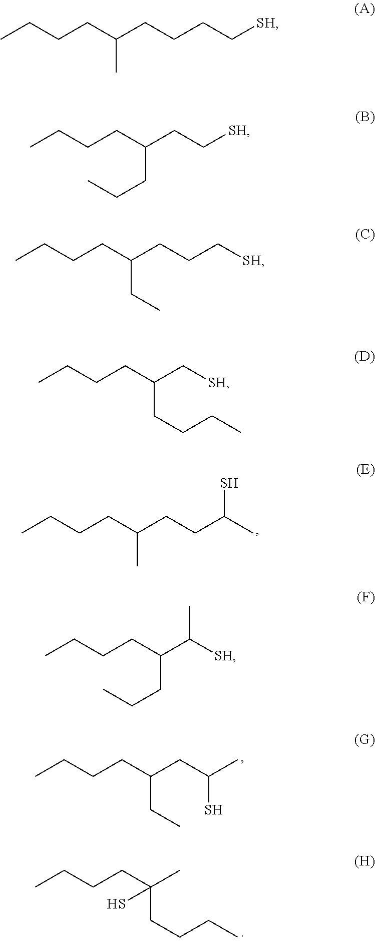

Mining chemical collector compositions can optionally include a third collector agent. More than one third collector agent can be utilized. The third collector agent can include a xanthate, a xanthic ester, a xanthogen formate, a dialkyl dithiophosphate, a phosphinate, a thionocarbamate, a hydrocarbon oil, or combinations thereof.

Xanthates of the third collector agent can include potassium amyl xanthate (PAX), sodium isopropyl xanthate (SIPX), or combinations thereof, among other suitable xanthates. The dialkyl dithiophosphate of the third collector agent can include any suitable dialkyl dithiophosphate, such as sodium diisobutyl dithiophosphate (DTP). Phosphinates of the third collector agent can include diisobutyldithiophosphinate, dithiophosphinate, or combinations thereof. In example of diisobutyldithiophosphinate can include Aerophine® 3418A which is an aqueous solution of sodium diisobutyldithiophosphinate. An example of dithiophosphinate can include Aerophine® 3422. Aerophine® is a blend of isopropyl ethyl thionocarbamate and dithiophosphinate.

The hydrocarbon oil of the third collector agent can include any suitable hydrocarbon oil. The hydrocarbon oil of the third collector agent can include an aliphatic hydrocarbon, a mixed aliphatic hydrocarbon, a paraffinic hydrocarbon, or combinations thereof, such as a mixture of C₁₃-C₁₆ isoalkanes. An example mixture of C₁₃-C₁₆ isoalkanes includes Orfom® MCX, commercially available from Chevron Phillips Chemical Company. Additionally, or alternatively, the hydrocarbon oil of the third collector agent can include a medium cycle oil. Medium cycle oils can include one or more decant (clarified) oils, light cycle oil, naphthalene, and polynuclear aromatics. An example medium cycle oil includes Orfom® MCO, commercially available from Chevron Phillips Chemical Company.

Mining chemical collector compositions of the present disclosure can include any suitable amount of the one or more alkylthiocarboxylic acids represented by formula (I). A total amount of the one or more alkylthiocarboxylic acids represented by formula (I) present in a mining chemical collector composition can be greater than 0 wt %, less than 99 wt %, or a combination thereof, such as in a range from about 1 wt % to about 80 wt %, such as from about 10 wt % to about 50 wt %, such as from about 20 wt % to about 30 wt % based on a total weight of the one or more alkylthiocarboxylic acids represented by formula (I), the optional one or more mercaptans represented by formula (II), and the optional third collector agent. The total weight of the one or more alkylthiocarboxylic acids represented by formula (I), the optional one or more mercaptans represented by formula (II), and the optional third collector agent is 100 wt %.

Mining chemical collector compositions of the present disclosure can include any suitable amount of the optional one or more mercaptans represented by formula (II). A total amount of the optional one or more mercaptans represented by formula (II) present in a mining chemical collector composition can be in a range from 0 wt % to about 99 wt %, such as in a range from about 1 wt % to about 80 wt %, such as from about 10 wt % to about 50 wt %, such as from about 10 wt % to about 30 wt % based on the total weight of the one or more alkylthiocarboxylic acids represented by formula (I), the optional one or more mercaptans represented by formula (II), and the optional third collector agent.

Mining chemical collector compositions of the present disclosure can include any suitable amount of the optional third collector agent. A total amount of the optional third collector agent can be in a range from 0 wt % to about 75 wt %, such as from greater than 0 wt % to about 75 wt %, such as from 5 wt % to about 50 wt %, such as from 10 wt % to about 40 wt %, based on the total weight of the one or more alkylthiocarboxylic acids represented by formula (I), the optional one or more mercaptans represented by formula (II), and the optional third collector agent.

An amount of the one or more alkylthiocarboxylic acids represented by formula (I) in mining chemical collector compositions described herein can be based on a metric ton of ore that it contacts during use. For example, an amount of the one or more alkylthiocarboxylic acids represented by formula (I) in mining chemical collector compositions described herein can be about 100 grams per metric ton of ore (g/t) or less, such as about 75 g/t or less, such as about 50 g/t or less, such as about 40 g/t or less, such as about 30 g/t or less, such as about 20 g/t or less, such as about 15 g/t or less, with a lower limit greater than 0 g/t of ore. Alternatively, an amount of the one or more alkylthiocarboxylic acids represented by formula (I) in mining chemical collector compositions described herein can be in a range from about 3 g/t to about 300 g/t, such as from about 6 g/t to about 150 g/t, such as from about 12 g/t to about 75 g/t, such as from about 24 g/t to about 37.5 g/t, such as about 30 g/t; or in a range from about 4 to about 80 g/t, such as from about 8 g/t to about 40 g/t, such as from about 16 g/t to about 25 g/t; or about 20 g/t. Alternatively, an amount of the one or more alkylthiocarboxylic acids represented by formula (I) in mining chemical collector compositions described herein can be in a range from about 1 g/t of the ore to about 100 g/t, such as from about 2 g/t to about 50 g/t, such as from about 4 g/t to about 25 g/t, such as from about 8 g/t to about 12.5 g/t; or about 10 g/t.

An amount of the optional one or more mercaptans represented by formula (II) present in mining chemical collector compositions described herein can be based on a metric ton of ore that it contacts during use. For example, an amount of the optional one or more mercaptans represented by formula (II) in mining chemical collector compositions described herein can be about 100 g/t or less, such as about 50 g/t or less, such as about 40 or less, such as about 30 g/t or less, such as about 20 g/t or less, such as about 15 g/t or less, such as about 10 g/t or less, such as about 5 g/t or less, with a lower limit of 0 g/t of ore or a lower limit of 0.5 g/t of ore. Alternatively, an amount of the optional one or more mercaptans represented by formula (II) in mining chemical collector compositions described herein can be in a range from about 0 g/t to about 50 g/t, such as from about 0.5 g/t of the ore to about 50 g/t, such as from about 1 g/t to about 25 g/t, such as from about 2 g/t to about 12.5 g/t, such as from about 4 g/t to about 10 g/t, such as from about 5 g/t to about 8 g/t, such as about 6 g/t or about 7 g/t; or in a range from about 4 g/t to about 7 g/t, such as from about 5 g/t to about 6 g/t; or about 5 g/t.

An amount of the optional third collector agent present in mining chemical collector compositions described herein can be based on a metric ton of ore that it contacts during use. For example, an amount of the optional third collector agent present in mining chemical collector compositions described herein can be 0 g/t or more, 100 g/t or less, or a combination thereof, such as in a range from about 1 g/t to about 100 g/t, such as from about 2 g/t to about 60 g/t, such as from about 4 g/t to about 40 g/t, such as from about 6 g/t to about 30 g/t, such as from about 8 g/t to about 25 g/t, such as from about 10 g/t to about 20 g/t, or about 10 g/t, or about 20 g/t. Alternatively, an amount of the optional third collector agent present in mining chemical collector compositions described herein can be in a range from about 2 g/t of the ore to about 200 g/t, such as from about 4 g/t to about 100 g/t, such as from about 8 g/t to about 50 g/t, such as from about 10 g/t to about 30 g/t, such as from about 15 g/t to about 25 g/t, or about 20 g/t.

Various weight ratios of the one or more alkylthiocarboxylic acids represented by formula (I), the optional one or more mercaptans represented by formula (II), and the optional third collector agent can be utilized in mining chemical collector compositions of the present disclosure.

For example, a weight ratio of the one or more alkylthiocarboxylic acids represented by formula (I) to the optional one or more mercaptans represented by formula (II) can be in a range from about 90:10 to about 10:90, such as from about 80:20 to about 20:80, such as from about 70:30 to about 30:70, such as from about 60:40 to about 40:60, such as about 50:50 (alkylthiocarboxylic acid:mercaptan), or in a range from about 11:1 to about 1:1, such as from about 10:1 to about 2:1, such as from about 9:1 to about 3:1, such as from about 8:1 to about 4:1, such as from about 7:1 to about 5:1, such as about 6:1, or from about 0.5:1 to about 4:1, such as from about 1:1 to about 3:1, such as from about 1.5:1 to about 2.5:1, such as from about 1.5:1 to about 2:1, or from about 2:1 to about 2.5:1, such as about 2:1, or about 2.5:1 (alkylthiocarboxylic acid:mercaptan). The weight ratio of the one or more alkylthiocarboxylic acids represented by formula (I) to the optional one or more mercaptans represented by formula (II) is based on the total amount of the one or more alkylthiocarboxylic acid represented by formula (I) present in a mining chemical collector composition to the total amount of the optional one or more mercaptans represented by formula (II) present in the mining chemical collector composition.

A weight ratio of the one or more alkylthiocarboxylic acids represented by formula (I) to the optional third collector agent can be in a range from about 10:1 to about 1:10, such as from about 7:1 to about 1:7, such as from about 4:1 to about 1:4, such as from about 3:1 to about 1:3, such as from about 2:1 to about 1:2, such as about 1:2, or about 2:1, or about 1.5:1 (alkylthiocarboxylic acid:third collector agent). The weight ratio of the one or more alkylthiocarboxylic acids represented by formula (I) to the optional third collector agent is based on the total amount of the one or more alkylthiocarboxylic acids represented by formula (I) present in a mining chemical collector composition to the total amount of the optional third collector agent present in the mining chemical collector composition.

A weight ratio of the optional one or more mercaptans represented by formula (II) to the optional third collector agent can be in a range from about 5:1 to about 1:5, such as from about 4:1 to about 1:4, such as from about 3:1 to about 1:3, such as from about 2:1 to about 1:2, or from about 5:95 to about 75:25, such as from about 10:90 to about 50:50, such as from about 15:85 to about 45:55, such as from about 20:80 to about 40:60, such as from about 25:75 to about 35:65, or from 15:85 to about 25:75, such as about 20:80 (mercaptan:third collector agent). The weight ratio of the optional one or more mercaptans represented by formula (II) to the optional third collector agent is based on the total amount of the optional one or more mercaptans represented by formula (II) present in a mining chemical collector composition to the total amount of the optional third collector agent present in the mining chemical collector composition.

Mining chemical collector compositions described herein can further include a pH control agent, a frothing agent, water, an organic solvent, or combinations thereof. Any suitable water can be used including, but not limited to, tap water, distilled water, well water, osmosis water, ground water, lake water, pond water, sea water, rainwater, or combinations thereof. Any suitable organic solvent can be used including a hydrocarbon solvent, an aromatic solvent, an alcohol solvent, or combination thereof. An amount of water or organic solvent present in mining chemical collector compositions described herein can be about 75 wt % or more, 95 wt % or more, 99 wt % or more, or in a range from about 75 wt % to about 99.99 wt %, such as from about 95 wt % to about 99.99 wt % based on a total weight of the mining chemical collector composition, the total weight of the mining chemical collector composition equal to 100 wt %.

pH control agents can be used, for example, to control the pH of the pulp during froth flotation. Any suitable pH control agent can be included in mining chemical collector compositions described herein. Illustrative, but non-limiting, examples of suitable pH control agents useful with aspects described herein can include lime, a carbonate compound (such as sodium carbonate), sulfuric acid, or combinations thereof. The pH of mining chemical collector compositions described herein can be in a range from about 4 to about 12, such as from about 6 to about 12, such as from about 7 to about 11, such as from about 7 to about 9, or from about 8 to about 12, such as from about 9 to about 11, such as from about 10 to about 11, such as about 10, or about 10.5, or about 11.

Frothing agents can be used, for example, to control the froth during froth flotation. Any suitable frothing agent can be included in mining chemical collector compositions described herein. Frothing agents useful with aspects described herein can include an alcohol, a hydrocarbon, or combinations thereof. Illustrative, but non-limiting, examples of suitable frothing agents can include: pine oil; an alcohol (for example, methyl isobutyl carbinol (MIBC)); a polyether alcohol (for example, NALFLOTE® 9837 and Cytec OREPREP® X-133); or combinations thereof. An example frothing agent that can be used in mining chemical collector compositions described herein can include Polyfroth® H10. Polyfroth® H10 includes a combination of products produced by the distillation of products from a 2-ethyl-1-hexanol manufacturing process (CAS NO: 68609-68-7). Another example frothing agent that can be used in mining chemical collector compositions of the present disclosure can include Quadrafroth 533 (CAS NO: 203588-70-9; a mixture of heavies made during the manufacturing process of 2-ethylhexanol).

Mining chemical collector compositions described herein can be utilized, for example, to recover one or more metals. Such recovery of one or more metals is described herein. Mining chemical collector compositions can be used as flotation compositions, for example, compositions used during performance of a flotation process.

Aspects of the present disclosure also generally relate to processes using compositions described herein. Such processes can include beneficiation of ore. Beneficiation of ore generally refers to various processes for separating a target mineral (for example, a valuable mineral) from waste minerals in ore, such as flotation processes or flotation procedures for the separation and/or recovery of a metal from ore. The metal can be recovered in any suitable form, for example, a metal-containing compound, a metal-containing mineral, a metal ion, an elemental metal, or combinations thereof.

An example flotation process or procedure for the recovery of a metal from an ore can include contacting an ore with a mining chemical collector composition described herein. Prior to contacting, the ore can be subjected to milling, crushing, grinding, wet grinding, or any suitable technique to make ore particles. During contacting, mining chemical collector compositions described herein can contact the ore particles. After contacting, selected ore particles can be recovered and/or other operations of a froth flotation process can be performed to, for example, recover a metal, a metal-containing mineral, a metal-containing compound, or combinations thereof.

Any suitable order of contacting any components of the mining chemical collector composition with the ore (or ore particles) can be used; and such mining chemical collector compositions can be in the form of solutions, slurries, blends, immiscible mixtures, or combinations thereof, among other forms. For example, a ground ore can be contacted with, in any order, an alkylthiocarboxylic acid represented by formula (I), an optional mercaptan represented by formula (II), an optional third collector agent (for example, an optional hydrocarbon oil), a pH control agent, a frothing agent, and water, forming a slurry. A second amount of water can be added to this slurry prior to the flotation process, resulting in a slurry of the ore in a collector composition comprising lower concentrations of the alkylthiocarboxylic acid, the optional mercaptan represented by formula (II), the optional third collector agent, the pH control agent, and the frothing agent.

In some aspects, which can be combined with other aspects, the ore can be contacted with the alkylthiocarboxylic acid represented by formula (I), the optional mercaptan represented by formula (II), and/or the optional third collector agent prior to being mixed with any water, pH control agent, or frothing agent. In some aspects, which can be combined with other aspects, the ore can be contacted with the alkylthiocarboxylic acid represented by formula (I), the optional mercaptan represented by formula (II), and/or the optional third collector agent (for example, hydrocarbon oil) prior to forming any slurry. Other suitable methods and orders of forming the mining chemical collector compositions, whether in the presence of the ore or not, are contemplated and are encompassed herein.

Aspects of the flotation processes described herein can generally include contacting the ore (for example, ore particles of a desired size) with the alkylthiocarboxylic acid represented by formula (I), the optional mercaptan represented by formula (II), and/or the optional third collector agent (for example, hydrocarbon oil) described herein before, during, or after: (i) any suitable step of the flotation processes described herein; (ii) any suitable step of a specified flotation process; and/or (iii) any suitable step of a flotation process known in the art but not specifically described herein. For example, the alkylthiocarboxylic acid represented by formula (I), the optional mercaptan represented by formula (II), and/or the optional third collector agent (for example, hydrocarbon oil) can be contacted with an ore or ore particles of a desired size:
(i) during grinding of the ore to a desired particle size (and optionally before, after, or with the addition of one or more other components of the mining chemical collector composition);
(ii) after grinding (for example, the alkylthiocarboxylic acid represented by formula (I), the optional mercaptan represented by formula (II), and/or the optional third collector agent can be added to a flotation cell containing the ore particles, and optionally before, after, or with the addition of one or more other components of the mining chemical collector composition);
(iii) during or after adjusting the pH of the material in the flotation cell (for example, the alkylthiocarboxylic acid represented by formula (I), the optional mercaptan represented by formula (II), and/or the optional third collector agent can be added to a flotation cell containing the ore particles, and optionally before, after, or with the addition of one or more other components of the mining chemical collector composition);
(iv) between and/or during froth removal stages (for example, the alkylthiocarboxylic acid represented by formula (I), the optional mercaptan represented by formula (II), and/or the optional third collector agent can be added to the flotation cell, and optionally before, after, or with the addition of one or more other components of the mining chemical collector composition);
(v) before, during, or after any other suitable step in a flotation process or procedure described herein, specified by a mine, and/or known in the art; or
(vi) combinations thereof.

It is contemplated that contacting the described alkylthiocarboxylic acid represented by formula (I), the optional mercaptan represented by formula (II), and/or the optional third collector agent with an ore or ore particles can include a series of additions of the alkylthiocarboxylic acid represented by formula (I), the optional mercaptan represented by formula (II), and/or the optional third collector agent (alone or in combination with one or more other components of the mining chemical collector composition) to a rod mill, a flotation cell, or any other suitable equipment containing the ore or ore particles.

Contacting the ore with one or more components of the mining chemical collector composition can be performed at least once. Contacting the ore with one or more components of the mining chemical collector composition can be performed at any suitable pH or pH range, such as a pH in a range from about 4 to about 12, such as from about 6 to about 12, such as from about 7 to about 11, such as from about 7 to about 9, or from about 8 to about 12, such as from about 9 to about 11, such as from about 10 to about 11, such as about 10, or about 10.5, or about 11.

Any suitable equipment and technique for the flotation recovery of various metals from ores can be used.

Generally, the metal recovered from the ore can include any suitable metal such as a transition metal, a post-transition metal, a metalloid, a rare earth metal (for example, a metal of the lanthanide series or actinide series), or combinations thereof. The metal can include one or more Group 3-16 metals, one or more Group 3-14 metals, one or more Group 3-12 metals, one or more Group 3-11 metals, or one or more Group 5-12 metals.

The metal can include scandium (Sc), yttrium (Y), lanthanum (La), actinium (Ac), titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), zinc (Zn), cadmium (Cd), mercury (Hg), aluminum (Al), gallium (Ga), indium (In), thallium (Tl), silicon (Si), germanium (Ge), tin (Sn), lead (Pb), arsenic (As), antimony (Sb), bismuth (Bi), tellurium (Te), polonium (Po), astatine (At), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), thorium (Th), Protactinium (Pa), uranium (U), neptunium (Np), plutonium (Pu), americium (Am), curium (Cm), berkelium (Bk), californium (Cf), einsteinium (Es), fermium (Fm), mendelevium (Md), nobelium (No), lawrencium (Lr), or combinations thereof.

In some aspects, which can be combined with other aspects, the metal can include Cu, Mo, Fe, Au, Ag, Pt, Ni, Pb, Zn, Co, Cr, Pd, Te, U, or combinations thereof, such as Cu, Mo, Fe, or combinations thereof. For example, copper can be recovered along with molybdenum and/or iron; molybdenum can be recovered along with copper and/or iron; iron can be recovered along with copper and/or molybdenum.

As described herein, the metal can be in the form of a compound or mineral. For example, the metal can be in the form of a metal sulfide, a metal oxide, or combinations thereof. Metal sulfides can include sulfides of the one or more aforementioned metals, such as a sulfide of Cu, Mo, Fe, Au, Ag, Pt, Ni, Pb, Zn, Co, Cr, Pd, Te, U, or combinations of such sulfides. The sulfide can include copper sulfide, molybdenum sulfide, iron sulfide, or combinations thereof. Metal oxides can include oxides of the one or more aforementioned metals, such as an oxide of Cu, Mo, Fe, Au, Ag, Pt, Ni, Pb, Zn, Co, Cr, Pd, Te, U, or combinations of such oxides. The oxide can include copper oxide, molybdenum oxide, iron oxide, or combinations thereof.

In contrast to conventional collector compositions, mining chemical collector compositions of the present disclosure are suitable for recovery of both metal sulfides and metal oxides together. For example, mining chemical collector compositions can be used to recover copper sulfides and copper oxides from ore. Additionally, or alternatively, mining chemical collector compositions can be used to recover molybdenum sulfides and molybdenum oxides from ore. Additionally, or alternatively, mining chemical collector compositions can be used to recover iron sulfides and molybdenum oxides from ore.

Any suitable amount of the mining chemical collector composition can be utilized in flotation recovery processes or procedures described herein. The alkylthiocarboxylic acid represented by formula (I), the optional mercaptan represented by formula (II), the optional third collector agent, and other optional components and the ore can be contacted at a selected weight ratio (in grams per metric ton of ore (g/t)). Such weight ratios are described herein.

An illustrative, but non-limiting, example of a flotation procedure can include the following operations: Water can be introduced to ore, followed by milling to produce ore particles of a certain size or size range. An amount of 1-50 g/t of a third collector agent (for example, a hydrocarbon oil) can be introduced to form a milled slurry. Additional water can then be added to the milled slurry. The pH of the slurry can then be adjusted to, for example, pH 10-12 by addition of a pH control agent to the slurry with stirring. An amount of 1-100 g/t of an alkylthiocarboxylic acid represented by formula (I) can be introduced to the slurry. Optionally, 1-100 g/t of an optional mercaptan represented by formula (II) can be introduced to the slurry. A frothing agent can then be added to the slurry. Flotation can then be performed to retrieve a concentrate and tailings. The concentrate and tailings can be dried for a suitable period at elevated temperature.

Flotation processes and mining chemical collector compositions described herein can be used with any suitable ore or mineral. The effectiveness of flotation processes and mining chemical collector compositions described herein can be beneficial when the ore or mineral includes copper, molybdenum, or a combination thereof. Illustrative, but non-limiting, examples of such ores can include chalcopyrite, chalcocite, molybdenite, and combinations thereof, among others.

Mining chemical collector compositions described herein can preferentially extract one or more metals relative to one or more different metals. For example, mining chemical collector compositions of the present disclosure can preferentially extract a copper-containing compound or mineral relative to an iron-containing compound or mineral. Additionally, or alternatively, mining chemical collector compositions of the present disclosure can preferentially extract a molybdenum-containing compound or mineral relative to an iron-containing compound or mineral. The selectivity against iron is a distinguishing feature of mining chemical collector compositions described herein relative to conventional compositions.

Mining chemical collector compositions of the present disclosure can form at least a portion of a copper collector composition. The copper collector composition can preferentially extract a copper-containing compound or mineral relative to a different metal-containing compound or mineral. Mining chemical collector compositions of the present disclosure can form at least a portion of a molybdenum collector composition. The molybdenum collector composition can preferentially extract a molybdenum-containing compound or mineral relative to a different metal-containing compound or mineral.

Flotation processes and mining chemical collector compositions described herein can provide superior results relative to conventional technologies in terms of, for example, recovery, grade recovered, selectivity, and/or mass recovered. Recovery, grade, selectivity, and mass recovered from an ore using aspects described herein relative to another composition can depend on the ore sample. “Recovery” refers to the amount (reported as a weight percentage) of a particular metal that is recovered after the flotation procedure compared to the amount of the particular metal in the original ore sample. “Grade” refers to the amount (reported as a weight percentage) of a particular metal in the product recovered from the flotation procedure. Mass recovery (reported as a weight percentage) refers to the mass of the concentrate divided by the mass of the initial sample.

Aspects described herein can enable improved copper recovery from an ore relative to conventional technologies. Any suitable percent recovery of copper from an ore can be achieved using aspects described herein. For example, a percent recovery of copper from an ore using a mining chemical collector composition described herein can be about 70 wt % or more, such as about 75 wt % or more, about 80 wt % or more, about 85 wt % or more, or about 90 wt % or more, etc., and up to about 99 wt %, up to about 95 wt %, etc. A percent recovery of copper from ore using a mining chemical collector composition described herein can be greater than a percent recovery of copper from ore using the composition without the alkylthiocarboxylic acid represented by formula (I) under the same flotation conditions, for example, by about 1 wt % or more, about 2 wt % or more, about 3 wt % or more, about 4 wt % or more, about 5 wt % or more, about 7 wt % or more, about 9 wt % or more, about 10 wt % or more, about 12 wt % or more, about 15 wt % or more, or about 20 wt % or more, etc., and up to about 99 wt %, up to about 75 wt %, or up to about 50 wt %, etc.

Aspects described herein can enable an improved copper grade recovered from an ore relative to conventional technologies. Any suitable percent grade of copper recovered from an ore can be achieved using aspects described herein. For example, a percent grade of copper recovered from an ore using a mining chemical collector composition described herein can be about 1 wt % or more, about 2 wt % or more, about 2.5 wt % or more, about 3 wt % or more, or about 4 wt % or more, etc., and up to about 30 wt %, up to about 20 wt %, or up to about 10 wt %, etc. A percent grade of copper recovered from an ore using a mining chemical collector composition described herein can be greater than that of the composition without the alkylthiocarboxylic acid represented by formula (I) under the same flotation conditions, for example, by about 1 wt % or more, about 2 wt % or more, or about 3 wt % or more, etc., and up to about 10 wt %, or up to about 5 wt %, etc.

Aspects described herein can enable improved molybdenum recovery from an ore relative to conventional technologies. Any suitable percent recovery of molybdenum from an ore can be achieved using aspects described herein. For example, a percent recovery of molybdenum from an ore using a mining chemical collector composition described herein can be about 50 wt % or more, about 55 wt % or more, about 60 wt % or more, about 65 wt % or more, or about 70 wt % or more, etc., and up to about 95 wt %, or up to about 90 wt %, etc. A percent recovery of molybdenum from ore using a mining chemical collector composition described herein can be greater than a percent recovery of molybdenum from ore using the composition without the alkylthiocarboxylic acid represented by formula (I) under the same flotation conditions, for example, by about 1 wt % or more, about 2 wt % or more, about 3 wt % or more, about 4 wt % or more, about 5 wt % or more, about 7 wt % or more, about 9 wt % or more, about 10 wt % or more, about 12 wt % or more, about 15 wt % or more, or about 20 wt % or more, etc., and up to about 99 wt %, up to about 75 wt %, or up to about 50 wt %, etc.

Aspects described herein can enable an improved molybdenum grade recovered from an ore relative to conventional technologies. Any suitable percent grade of molybdenum recovered from an ore can be achieved using aspects described herein. For example, a percent grade of molybdenum recovered from an ore using a mining chemical collector composition described herein can be about 0.05 wt % or more, about 0.08 wt % or more, about 0.1 wt % or more, or about 0.12 wt % or more, or about 0.14 wt % or more, or about 0.15 wt % or more, or about 0.16 wt % or more, etc., and up to about 3 wt %, or up to about 2 wt %, etc. A percent grade of molybdenum recovered from an ore using a mining chemical collector composition described herein can be greater than that of the composition without the alkylthiocarboxylic acid represented by formula (I) under the same flotation conditions, for example, by about 1.1× or more, about 1.2× or more, about 1.5× or more, about 2× or more, or about 3× or more, etc., and up to about 3×, or up to about 2.5×, etc.

As described herein, aspects of the present disclosure can be selective against iron. For example, aspects described herein can be more selective to extract copper-containing minerals or compounds and/or molybdenum-containing minerals or compounds over iron-containing minerals or compounds. Aspects described herein can have improved selectivity against iron compared to conventional technologies.

Any suitable percent recovery of iron from an ore can be achieved using aspects described herein. For example, a percent recovery of iron from an ore using a mining chemical collector composition described herein can be about 5 wt % or more, about 10 wt % or more, about 20 wt % or more, about 25 wt % or more, about 30 wt % or more, or about 35 wt % or more, etc., and up to about 60 wt %, up to about 50 wt %, up to about 40 wt %, up to about 30 wt %, or up to about 20 wt %, etc. A percent recovery of iron from an ore using a mining chemical collector composition described herein can be lower than a percent recovery of iron from ore using the composition without the alkylthiocarboxylic acid represented by formula (I) under the same flotation conditions, for example, by about 1 wt % or more, about 2 wt % or more, about 3 wt % or more, about 4 wt % or more, about 5 wt % or more, about 7 wt % or more, about 9 wt % or more, about 10 wt % or more, about 12 wt % or more, about 15 wt % or more, or about 20 wt % or more, etc., and up to about 50 wt %, up to about 30 wt %, or up to about 10 wt %, etc.

A percent recovery of iron from an ore using a mining chemical collector composition described herein can be lower than a percent recovery of iron from ore using a non-selective collector (such as a xanthate) under the same flotation conditions, for example, by about 1 wt % or more, about 2 wt % or more, about 3 wt % or more, about 4 wt % or more, about 5 wt % or more, about 7 wt % or more, about 9 wt % or more, about 10 wt % or more, about 12 wt % or more, about 15 wt % or more, or about 20 wt % or more, etc., and up to about 50 wt %, up to about 30 wt %, up to about 20 wt %, or up to about 10 wt %, etc.

Any suitable percent grade of iron recovered from an ore can be achieved using aspects described herein. For example, a percent grade of iron recovered from an ore using a mining chemical collector composition described herein can be about 5 wt % or more, about 6 wt % or more, about 7 wt % or more, about 8 wt % or more, about 9 wt % or more, about 10 wt % or more, about 11 wt % or more, or about 12 wt % or more, etc., and up to about 40 wt %, up to about 30 wt %, up to about 20 wt %, or up to about 10 wt %, etc. A percent grade of iron recovered from an ore using a mining chemical collector composition described herein can be greater than that of the composition without the alkylthiocarboxylic acid represented by formula (I) under the same flotation conditions, for example, by about 1 wt % or more, about 2 wt % or more, about 3 wt % or more, or about 4 wt % or more, etc., and up to about 15 wt %, up to about 10 wt %, or up to about 5 wt %, etc.

Aspects described herein can also provide a lower percent mass recovered from the ore (e.g., a lower total mass recovered from the ore) relative to conventional approaches. Lower percent masses recovered from the ore can be more efficient for downstream processing relative to higher percent masses recovered. Any suitable mass recovery from the ore can be achieved using aspects described herein. A mass recovery from the ore using a mining chemical collector composition described herein can be greater than 0 wt % and up to about 10 wt % or less, such as about 9 wt % or less, such as about 8 wt % or less, such as about 7 wt % or less, such as about 6 wt % or less, such as about 5 wt % or less, etc.

Aspects of the present disclosure can be further understood by the following non-limiting examples. The following non-limiting examples are put forth so as to provide those of ordinary skill in the art with a complete disclosure and description of how to make and use aspects of the present disclosure, and are not intended to limit the scope of aspects of the present disclosure.

### EXAMPLES

The recovery of metals utilizing mining chemical collector compositions described herein were evaluated. Various advantages of mining chemical collector compositions described herein are illustrated. For example, the Examples show the effectiveness of using an alkylthiocarboxylic acid represented by formula (I) in mining chemical collector compositions. The Examples also demonstrate a synergistic effect, and not merely an additive effect, between the alkylthiocarboxylic acid represented by formula (I) and a mercaptan represented by formula (II), when a diminished result would have been expected. Other advantages were also observed.

### Materials

Alkylthiocarboxylic acids represented by formula (I) and used for the examples included the following alkylthiocarboxylic acids: CL-1 (R¹ of formula (I) is hexyl); CL-2 (R¹ of formula (I) is cyclohexyl); CL-3 (R¹ of formula (I) is octyl); CL-4 (R¹ of formula (I) is decyl); CL-5 (a mixture of compounds, wherein each R¹ of formula (I) of the mixture of compounds is a branched C10 hydrocarbyl); CL-6 (R¹ of formula (I) is hexyl, and n of formula (I) is 2).

Mercaptans represented by formula (II) and used for the examples included Orfom® CO100 (n-dodecyl mercaptan), commercially available from Chevron Phillips Chemical Company.

Other collector agents included sodium isopropyl xanthate (SIPX) and sodium dithiophosphate (DTP).

Hydrocarbon oils used for the examples included Orfom® MCO hydrocarbon oil. Frothing agents used for the examples included Quadrafroth 533. The hydrocarbon oil and frothing agent are described herein.

### Example 1

A first set of experiments was completed using a copper-molybdenum ore from the American southwest (Mine A). The ore matrix contained 0.34% copper, 0.12% molybdenum, and 1.94% iron, which was measured using x-ray fluorescence (XRF) without calibration adjustments for the ore matrix. An amount of 1 kg of the ore was added to 650 mL of tap water and milled to produce a flotation product at 75% pass 70 mesh (P75=210 μm). For all samples, 20 g/t of Orfom® MCO hydrocarbon oil collector was added to the grind.

The milled slurry was placed in a 2 L Denver flotation cell and tap water was added to reach the desired flotation volume. The solids amount was about 33% solids by weight. With mixing, the pH was adjusted to 11 using lime. The collectors (Orfom® CO100 and CL-1 to CL-6) were added according to Table 1A and conditioned for 1 minute, followed by addition of 20 μL of frothing agent (Quadrafroth 533) prior to the float. The flotations were completed for a total of 6 minutes. One rougher concentrate sample and one tailings sample were collected by filtration and dried overnight at 160° F. (71° C.). The samples were ground to a fine powder and analyzed by XRF.

Table 1A shows the amount and type of selected collectors for the example mining chemical collector compositions (Ex. 1-1, Ex. 1-2, Ex. 1-3, Ex. 1-4, Ex. 1-5, Ex. 1-6, Ex. 1-7, and Ex. 1-8) and the comparative composition (C.Ex. 1-1). The amounts of the collector are based on grams per metric ton of ore (g/t).

TABLE 1A

| | Collector, g/t | | | | | | | |
|---|---|---|---|---|---|---|---|---|
| Sample | CO100 | CL-1 | CL-2 | CL-3 | CL-4 | CL-5 | CL-6 | MCO |
| C. Ex. 1-1 | 5 | — | — | — | — | — | — | 20 |
| Ex. 1-1 | 5 | 30 | — | — | — | — | — | 20 |
| Ex. 1-2 | 5 | — | 30 | — | — | — | — | 20 |
| Ex. 1-3 | 5 | — | — | 30 | — | — | — | 20 |
| Ex. 1-4 | 5 | — | — | — | 30 | — | — | 20 |
| Ex. 1-5 | 5 | — | — | — | — | 30 | — | 20 |
| Ex. 1-6 | 5 | — | — | — | — | — | 30 | 20 |
| Ex. 1-7 | — | 20 | — | — | — | — | — | 20 |
| Ex. 1-8 | — | — | — | 20 | — | — | — | 20 |

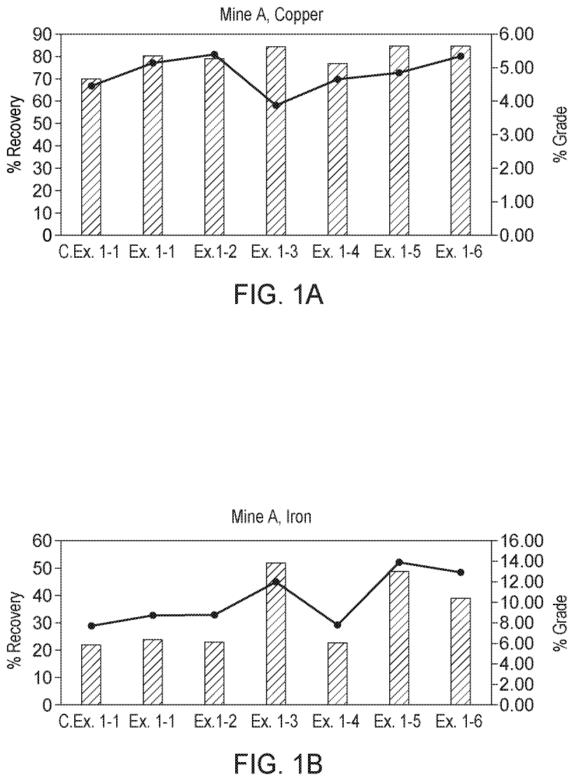

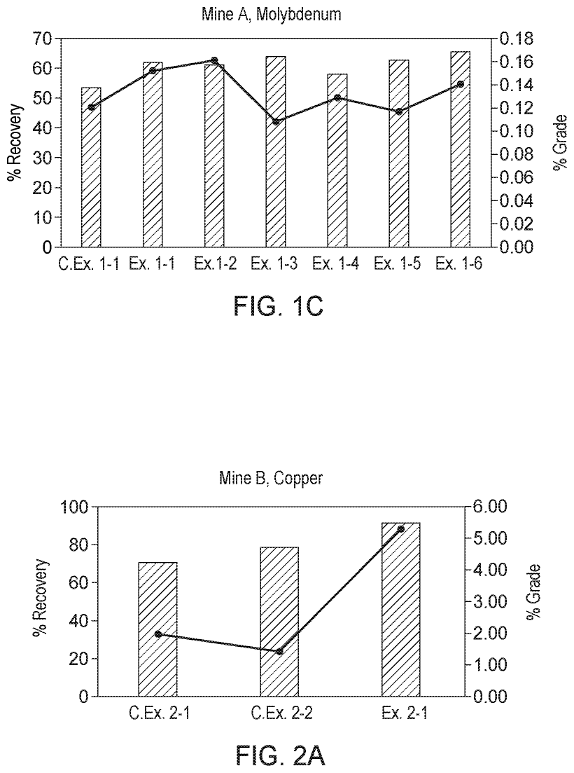

Table 1B shows % recovery (reported as a wt %), % grade (reported as a wt %), and % mass recovered (reported as a wt %) using the example and comparative compositions. FIGS. 1A-1C show selected results for metal recovery using the example and comparative compositions, with the bar chart illustrating % recovery and the line chart illustrating % grade. Exs. 1-1 to 1-6 included an alkylthiocarboxylic acid represented by formula (I) and a mercaptan represented by formula (II). Exs. 1-7 and 1-8 included an alkylthiocarboxylic acid represented by formula (I).

TABLE 1B

| | | | | | | | Mass |
|---|---|---|---|---|---|---|---|
| | wt % Recovery | | | wt % Grade | | | Recovered, |
| Sample | Cu | Fe | Mo | Cu | Fe | Mo | wt % |
| C. Ex. 1-1 | 70 | 22 | 54 | 4.45 | 7.73 | 0.12 | 6 |
| Ex. 1-1 | 80 | 24 | 62 | 5.13 | 8.74 | 0.15 | 6 |
| Ex. 1-2 | 79 | 23 | 61 | 5.36 | 8.78 | 0.16 | 5 |
| Ex. 1-3 | 84 | 52 | 64 | 3.87 | 11.96 | 0.11 | 9 |
| Ex. 1-4 | 77 | 23 | 58 | 4.65 | 7.87 | 0.13 | 6 |
| Ex. 1-5 | 85 | 49 | 63 | 4.83 | 13.79 | 0.12 | 7 |
| Ex. 1-6 | 85 | 39 | 65 | 5.33 | 12.97 | 0.14 | 6 |
| Ex. 1-7 | 65 | 15 | 56 | 3.88 | 5.62 | 0.14 | 5 |
| Ex. 1-8 | 63 | 16 | 64 | 3.63 | 5.90 | 0.14 | 6 |

The data in Table 1B demonstrates the improvement of Cu and Mo recovery using mining chemical collector compositions of the present disclosure relative to conventional compositions. For example, Exs. 1-1 to 1-6 showed a higher recovery of both Cu (about 77 wt % to about 85 wt %) and Mo (about 58 wt % to about 65 wt %) than the comparative examples (Cu: 70 wt %; Mo: 54 wt %). This result indicates the beneficial effect of using a blend of alkylthiocarboxylic acid represented by formula (I) with an optional mercaptan represented by formula (II) instead of a mercaptan alone. The data also shows that alkylthiocarboxylic acids described herein can be used to recover both Cu and Mo. For example, Ex. 1-7 and 1-8 recovered greater than 60 wt % Cu; both Exs. 1-7 and 1-8 also showed higher recovery of Mo than C.Ex. 1-1.

The data also indicates that alkylthiocarboxylic acids represented by formula (I) can be used for improved selectivity against Fe relative to the use of a mercaptan. For example, the recovery of Fe decreased from 22 wt % (C.Ex. 1-1: mercaptan) to about 15 wt % (Ex. 1-7: alkylthiocarboxylic acid) and about 16 wt % (Ex. 1-8: alkylthiocarboxylic acid). Even though the selectivity against Fe stayed about the same level for Ex. 1-1, Ex. 1-2, and Ex. 1-4 as C.Ex. 1-1, the significant improvement in Cu and Mo recovery in the inventive examples demonstrates that such compositions have an overall improved balance of Cu recovery, Mo recovery, and selectivity against Fe. In addition, the grades of Cu, Mo, and Fe metals recovered improved for each of Exs. 1-1, 1-2, 1-4, 1-5, and 1-6.

The data of Table 1B also demonstrates the synergy observed between the alkylthiocarboxylic acids represented by formula (I) and the mercaptan represented by formula (II). The synergy allowed, for example, improved metal recovery.

For example, C.Ex. 1-1 (with the CO100 mercaptan) had a Cu recovery of 70 wt %. Ex. 1-7 (with the CL-1 alkylthiocarboxylic acid) also demonstrated Cu recovery, but a slightly lower value of about 65 wt %. One would have expected that the combination of the mercaptan and the alkylthiocarboxylic acid would have a lower Cu recovery, for example, lower than 70 wt %. However, Ex. 1-1 (with the CO100 mercaptan and the CL-1 alkylthiocarboxylic acid) performed better for Cu recovery, and unexpectedly increased the Cu recovery to about 80 wt % even with the relatively lower amount of mercaptan. Instead of a diminished result that would have been expected, a greater than expected result was observed using mining chemical collector compositions of the present disclosure. This greater than expected result was not merely additive, but was synergistic.

Exs. 1-1 to 1-6 demonstrate that alkylthiocarboxylic acids represented by formula (I) and mercaptans represented by formula (II) are an effective collector combination for extracting metal from ore. Exs. 1-7 and 1-8 demonstrate that alkylthiocarboxylic acids represented by formula (I) are also useful in extracting metal from ore. and Overall, the mining chemical collector compositions of the present disclosure perform significantly better than conventional compositions.

### Example 2

A second set of experiments was completed using a copper-molybdenum ore from the American southwest (Mine B). The ore matrix contained 0.26% copper, 0.008% molybdenum, and 1.6% iron, which was measured using XRF without adjustment for the ore matrix. An amount of 1 kg of the ore was added to 650 mL of tap water and milled to produce a flotation product at 75% pass 70 mesh (P75=210 μm). For all samples, 20 g/t of Orfom® MCO hydrocarbon oil collector was added to the grind.

The milled slurry was placed in a 2 L Denver flotation cell and tap water was added to reach the desired flotation volume. The solids amount was about 33% solids by weight. With mixing, the pH was adjusted to 10.5 using lime. The collectors (Orfom® CO100, CL-6, or DTP/SIPX) were added according to Table 2A and conditioned for 1 minute, followed by addition of 30 μL of Quadrafroth 533 frothing agent prior to the float. The flotations were completed for a total of 6 minutes. One rougher concentrate sample and one tailings sample were collected by filtration and dried overnight at 160° F. The samples were ground to a fine powder and analyzed by XRF.

Table 2A shows the amount and type of selected collectors for the example mining chemical collector composition (Ex. 2-1) and comparative compositions (C.Ex. 2-1 and C.Ex. 2-2). The amounts of collector are based on grams per metric ton of ore (g/t).

TABLE 2A

Collector, g/t

| Sample | CO100 | CL-6 | DTP/SIPX | MCO |
|---|---|---|---|---|
| Ex. 2-1 | 5 | 10 | — | 20 |
| C. Ex. 2-1 | 5 | — | — | 20 |
| C. Ex. 2-2 | 5 | — | 7/2 | 20 |

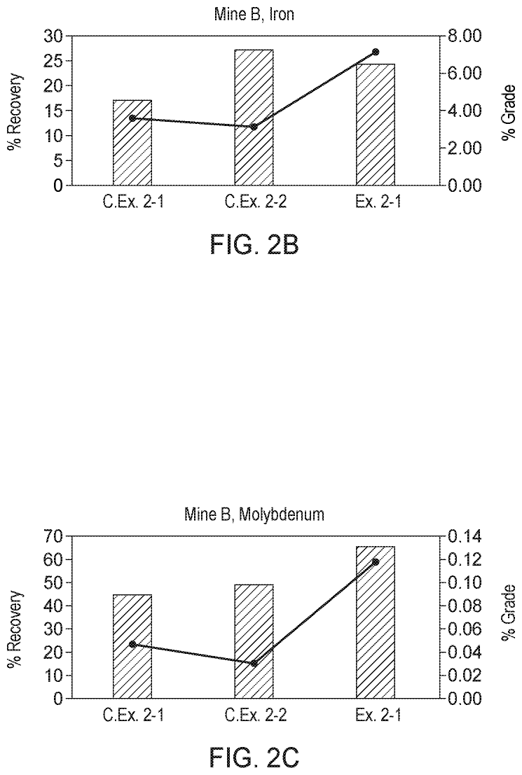

Table 2B shows % recovery (reported as a wt %), % grade (reported as a wt %), and % mass recovered (reported as a wt %) using the example and comparative compositions. DTP/SIPX refers to a blend of DTP and SIPX. FIGS. 2A-2C show selected results for metal recovery using the example and comparative compositions, with the bar chart illustrating % recovery and the line chart illustrating % grade.

TABLE 2B

| | | | | | | | Mass |
|---|---|---|---|---|---|---|---|
| | wt % Recovery | | | wt % Grade | | | Recovered, |
| Sample | Cu | Fe | Mo | Cu | Fe | Mo | wt % |
| Ex. 2-1 | 91 | 24 | 65 | 5.33 | 7.14 | 0.12 | 5 |
| C. Ex. 2-1 | 70 | 17 | 45 | 1.97 | 3.59 | 0.05 | 9 |
| C. Ex. 2-2 | 79 | 27 | 49 | 1.41 | 3.14 | 0.03 | 13 |

The data in Table 2B demonstrates that mining chemical collector compositions described herein, including a combination of an alkylthiocarboxylic acid represented by formula (I) and a mercaptan, have significantly improved collector properties over conventional compositions. For example, Ex. 2-1 shows a significant improvement in Cu and Mo recovery over C.Exs. 2-1 and 2-2, increasing Cu recovery from 79 wt % or 70 wt % (C.Ex. 2-1 and C.Ex. 2-2) to about 91 wt % (Ex. 2-1) and increasing Mo recovery from 49 wt % or 45 wt % (C.Ex. 2-1 and C.Ex. 2-2) to 65 wt % (Ex. 2-1). The results demonstrate the advantageous effect of using an alkylthiocarboxylic acid represented by formula (I) with a mercaptan represented by formula (II) over both the comparative composition having a mercaptan (C.Ex. 2-1) and the comparative composition having a xanthate and dithiophosphate blend with a mercaptan (C.Ex. 2-2).

The grades of Cu and Mo recovered from the ore using Ex. 2-1 were also significantly improved over the comparative examples, indicating another advantageous effect using the combination of the alkylthiocarboxylic acid represented by formula (I) and the mercaptan represented by formula (II). Here, the grade of Cu recovered from the ore improved from 1.41 wt % (C.Ex. 2-2) and 1.97 wt % (C.Ex. 2-1) to about 5.33 wt % (Ex. 2-1). The grade of Cu recovered from the ore using Ex. 2-1 (Cu grade: 5.33 wt %) was more than triple that of the comparative composition having a xanthate and dithiophosphate blend with a mercaptan (C.Ex. 2-2; Cu grade: 1.41 wt %). Also, the grade of Cu recovered from the ore using Example 2-1 (Cu grade: 5.33 wt %) was more than double the grade of Cu recovered from the ore using the mercaptan (C.Ex. 2-1; Cu grade: 1.97 wt %). Similar results were observed for the grade of Mo recovered.

The data also shows that the use of an alkylthiocarboxylic acid represented by formula (I) resulted in improved selectivity against Fe relative to the use of a xanthate and dithiophosphate blend (C.Ex. 2-2). For example, the recovery of Fe from the ore decreased from 27 wt % (C.Ex. 2-2: xanthate/dithiophosphate blend and mercaptan) to about 24 wt % (Ex. 2-1) with use of an alkylthiocarboxylic acid represented by formula (I) and a mercaptan represented by formula (II).

The grade of Fe recovered from the ore also significantly improved using mining chemical collector compositions of the present disclosure. Here, the grade of Fe recovered using the comparative examples was determined to be 3.59 wt % (C.Ex. 2-1) and 3.14 wt % (C.Ex. 2-2). In contrast, the grade of Fe recovered using Ex. 2-1 was determined to be about 7.14 wt % or more, which is approximately twice that of C.Ex. 2-1 (3.59 wt %) and more than doubling that of C.Ex. 2-2 (3.14 wt %).

Further, the percent mass recovered from the ore (a total mass recovered from the ore) using Ex. 2-1 was determined to be about 5 wt %, while the mass recovered using C.Ex. 2-1 (mercaptan) and C.Ex. 2-2 (xanthate/dithiophosphate blend and mercaptan) was determined to be significantly higher at 9 wt % and 13 wt %, respectively. This substantial decrease in mass recovered and much higher grades using Ex. 2-1 indicates that downstream processing efficiency of the recovered mass using the example compositions should be improved over the recovered mass from C.Ex. 2-1 and C.Ex. 2-2.

Overall, Example 2 shows that mining chemical collector compositions of the present disclosure perform significantly better than conventional compositions.

### Example 3: Synthesis of Alkylthiocarboxylic Acids

In Example 3, various thioether carboxylic acids were formed as example functionalized organic sulfides. Scheme 3-1 shows a reaction diagram for Example 3 between an olefin represented by formula (I) and a mercaptan represented by formula (II) to form a thioether carboxylic acid.

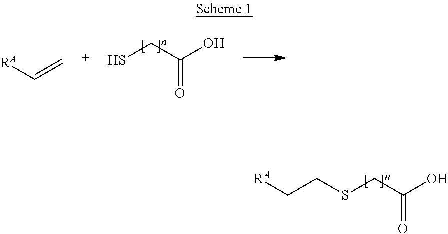

A 500 mL 3-necked flask equipped with a condenser and a thermocouple was sparged with nitrogen. Olefin was then added. The olefin was stirred under nitrogen at the desired temperature. A molar excess of the olefin was used, and specifically, a 5:1 olefin:mercaptan molar ratio was utilized. Thioglycolic acid or 3-mercaptopropanoic acid was slowly added. When the reaction was complete, a distillation column was inserted between the condenser and the 3-necked flask. Nitrogen was sparged through the reaction mixture and the temperature was slowly raised until excess (unreacted) olefin was separated. Once unreacted olefin had been completely separated, GC-MS and NMR analysis were performed on the final product.

Table 3 shows the results for the reaction to form various thioether carboxylic acids as examples of functionalized organic sulfides. The structure of products CL-1, CL-2, CL-3, CL-4, CL-5, and CL-6 are provided below the table along with ¹H and ¹³C NMR data.

TABLE 3

| | | | Temp., | |
|---|---|---|---|---|
| Ex. No. | Olefin | Mercaptan | ° C. | Product |
| Ex. 3-1 | 1-hexene | thioglycolic acid | 25 | CL-1 |
| Ex. 3-2 | cyclohexene | thioglycolic acid | 25 | CL-2 |
| Ex. 3-3 | 1-octene | thioglycolic acid | 50 | CL-3 |
| Ex. 3-4 | 1-decene | thioglycolic acid | 50 | CL-4 |
| Ex. 3-5 | mixture of branched | thioglycolic acid | 25 | CL-5 |
| | C10 monoolefins | | | |
| Ex. 3-6 | 1-hexene | 3-mercaptopropanoic | 50 | CL-6 |
| | | acid | | |

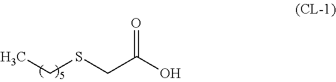

2-(hexylthio)acetic acid (CL-1): ¹H NMR (400 MHz, CDCl₃) δ 3.25 (s, 2H), 2.66 (t, J=7.4 Hz, 2H), 1.60 (p, J=7.2 Hz, 2H), 1.39 (dt, J=14.1, 6.9 Hz, 2H), 1.30 (s, 4H), 0.95-0.85 (m, 3H). ¹³C NMR (101 MHz, CDCl₃) δ 177.35, 77.55, 77.23, 76.91, 33.58, 32.88, 31.44, 28.94, 28.47, 22.59, 14.08.

Distortionless Enhancement by Polarization Transfer (DEPT) NMR was used to determine the multiplicity of various carbon atoms in CL-1. A single CH₃ signal in the DEPT spectrum was observed, validating the linear structure of the 2-(hexylthio)acetic acid obtained.

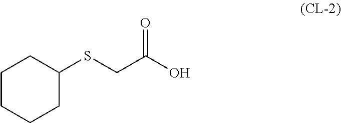 [SMILES: O=C(O)CSC1CCCCC1]

2-(cyclohexylthio)acetic acid (CL-2): ¹H NMR (400 MHz, CDCl₃) δ 3.29 (s, 2H), 2.90-2.73 (m, 1H), 2.07-1.93 (m, 2H), 1.78 (s, 2H), 1.63 (d, J=10.9 Hz, 1H), 1.40-1.17 (m, 5H). ¹³C NMR (101 MHz, CDCl₃) δ 177.33, 77.32, 77.00, 76.68, 43.84, 32.80, 31.62, 25.71, 25.54.

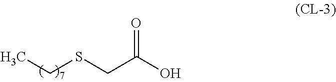

2-(octylthio)acetic acid (CL-3): ¹H NMR (400 MHz, CDCl₃) δ 3.26 (s, 1H), 2.66 (t, J=6.8 Hz, 1H), 1.61 (p, J=7.2 Hz, 2H), 1.44-1.34 (m, 2H), 1.34-1.26 (m, 7H), 0.92-0.84 (m, 3H). ¹³C NMR (101 MHz, CDCl₃) δ 177.16, 77.32, 77.00, 76.68, 33.38, 32.68, 31.68, 29.04, 29.02, 28.78, 28.62, 22.52, 13.96.

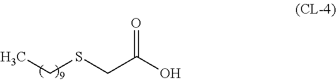

2-(decylthio)acetic acid (CL-4): ¹H NMR (400 MHz, CDCl₃) δ 3.25 (s, 2H), 2.71-2.61 (m, 2H), 1.60 (p, J=7.2 Hz, 2H), 1.44-1.33 (m, 2H), 1.26 (s, 12H), 0.94-0.80 (m, 3H). ¹³C NMR (101 MHz, CDCl₃) δ 177.15, 33.37, 32.69, 31.78, 29.44, 29.39, 29.20, 29.08, 28.79, 28.63, 22.57, 13.98.

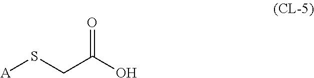

2-(mixed-decylthio)acetic acid (CL-5): “A” in structure CL-5 represents a branched C10 hydrocarbyl. ¹H NMR (400 MHz, CDCl₃) δ 3.25 (s, 2H), 2.66 (t, J=7.4 Hz, 2H), 1.67-1.50 (m, 2H), 1.26 (s, 13H), 1.10 (d, J=47.5 Hz, 2H), 0.79 (s, 7H). ¹³C NMR (101 MHz, CDCl₃) δ 177.41, 77.55, 77.23, 76.91, 38.69, 36.82, 36.75, 36.69, 35.83, 33.69, 33.63, 33.44, 33.11, 33.03, 32.97, 32.94, 32.92, 32.89, 32.77, 32.45, 32.04, 31.94, 30.59, 29.69, 29.64, 29.44, 29.38, 29.33, 29.29, 29.28, 29.04, 28.87, 26.36, 26.33, 26.29, 25.93, 23.25, 23.20, 23.16, 22.82, 22.78, 19.82, 19.76, 14.56, 14.29, 14.24, 14.22, 10.94.

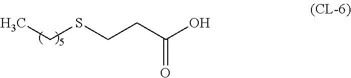

3-(hexylthio)propanoic acid (CL-6): ¹H NMR (400 MHz, CDCl₃) δ 2.85-2.74 (m, 2H), 2.71-2.62 (m, 2H), 2.58-2.48 (m, 2H), 1.58 (p, J=7.3 Hz, 2H), 1.44-1.33 (m, 2H), 1.23 (s, 4H), 0.89 (t, J=6.9 Hz, 3H). ¹³C NMR (101 MHz, CDCl₃) δ 178.35, 34.69, 32.04, 31.26, 29.34, 28.38, 26.43, 22.39, 13.87.

In all cases, the desired product was obtained. For Ex. 3-3 and Ex. 3-4, complete solidification of the reaction mixture was observed at 25° C. which prompted the use of a higher reaction temperature of 50° C. When using 3-mercaptopropanoic acid (Ex. 3-6) instead of thioglycolic acid, the reaction progressed more slowly at 25° C. Therefore, a higher reaction temperature of 50° C. was utilized.

The results demonstrated that the reaction between a carboxylic-acid containing mercaptan (thioglycolic acid or 3-mercaptopropanoic acid) and a variety of olefins occurs through anti-Markovnikov addition of the mercaptan adding to the less substituted carbon of the olefin.

Aspects of the present disclosure generally relate to a new class of mining chemical collector compositions that include an alkylthiocarboxylic acid represented by formula (I). The mining chemical collector compositions can be used in processes for recovering a metal from an ore. Mining chemical collector compositions described herein are characterized as having superior recovery of Cu and Mo, as well as superior grades of metal recovered, relative to conventional collector compositions. Mining chemical collector compositions described herein also have excellent selectivity against Fe. Mining chemical collector compositions of the present disclosure are also very effective at recovering mixed sulfide/oxide minerals unlike state-of-the-art collector compositions.

### Aspects of the Disclosure

The present disclosure provides, among others, the following aspects, each of which can be considered as optionally including any alternate aspects:

Aspect 1. A mining chemical collector composition, comprising:
a first collector agent comprising one or more alkylthiocarboxylic acids, each alkylthiocarboxylic acid represented by formula (I):

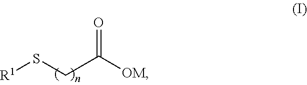

wherein: R¹ of formula (I) is a C4-C20 hydrocarbyl; n of formula (I) is 1 or 2; and M of formula (I) is hydrogen (H), Li, Na, K, Rb, or Cs.

Aspect 2. The mining chemical collector composition according to Aspect 1, wherein R¹ of formula (I) is a linear or branched, acyclic or cyclic C4-C20 hydrocarbyl.

Aspect 3. The mining chemical collector composition according to any one of the preceding Aspects, wherein R¹ of formula (I) contains at least one unsaturated bond.

Aspect 4. The mining chemical collector composition according to any one of the preceding Aspects, wherein R¹ is a C5-C18 hydrocarbyl, such as a C6-C16 hydrocarbyl, such as n-hexyl, cyclohexyl, n-octyl, n-decyl, or a branched C10 hydrocarbyl.

Aspect 5. The mining chemical collector composition according to Aspect 4, wherein the branched C10 hydrocarbyl is derived from a branched C10 monoolefin comprising 5-methyl-1-nonene (represented by structure A), 3-propyl-1-heptene (represented by structure B), 4-ethyl-1-octene, (represented by structure C), 2-butyl-1-hexene (represented by structure D), or combinations thereof:

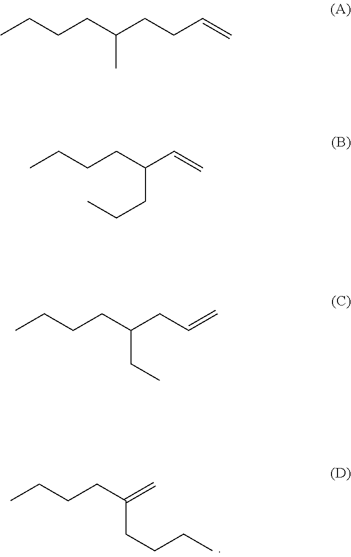

Aspect 6. The mining chemical collector composition according to any one of the preceding Aspects, wherein R¹ of formula (I) is hexyl, and n of formula (I) is 2.

Aspect 7. The mining chemical collector composition according to any one of the preceding Aspects, wherein the one or more alkylthiocarboxylic acids represented by formula (I) comprise one or more alkylthiocarboxylic acids represented by formula (I-A), formula (I-B), formula (I-C), formula (I-D), formula (I-E), formula (I-F), formula (I-G), formula (I-H), or combinations thereof:

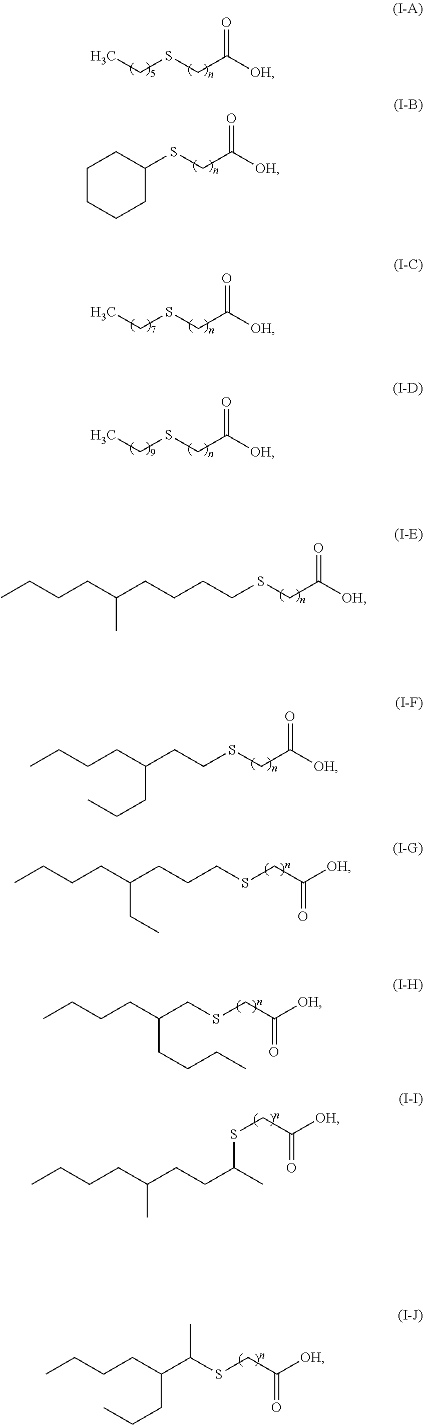

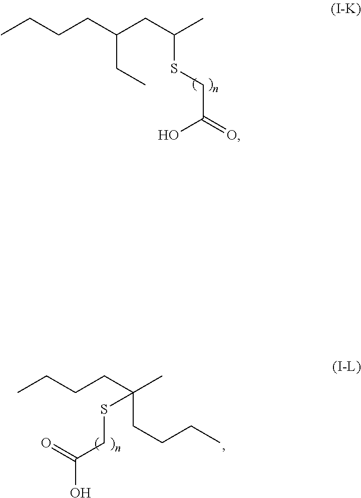

or combinations thereof,
wherein n of formulas (I-A), (I-B), (I-C), (I-D), (I-E), (I-F), (I-G), (I-H), (I-I), (I-J), (I-K), and (I-L) is, independently, 1 or 2.

Aspect 8. The mining chemical collector composition according to any one of the preceding Aspects, wherein the one or more alkylthiocarboxylic acids represented by formula (I) comprise:

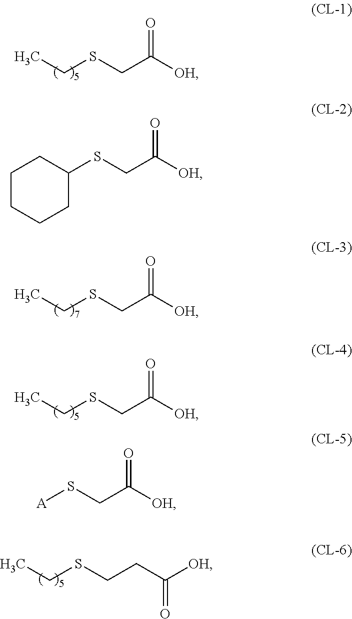

combinations thereof, and
wherein A of structure CL-5 is a branched C10 hydrocarbyl.

Aspect 9. The mining chemical collector composition according to Aspect 8, wherein:
the one or more alkylthiocarboxylic acids represented by formula (I) comprise structure CL-5; and
CL-5 comprises one or more of the following compounds:

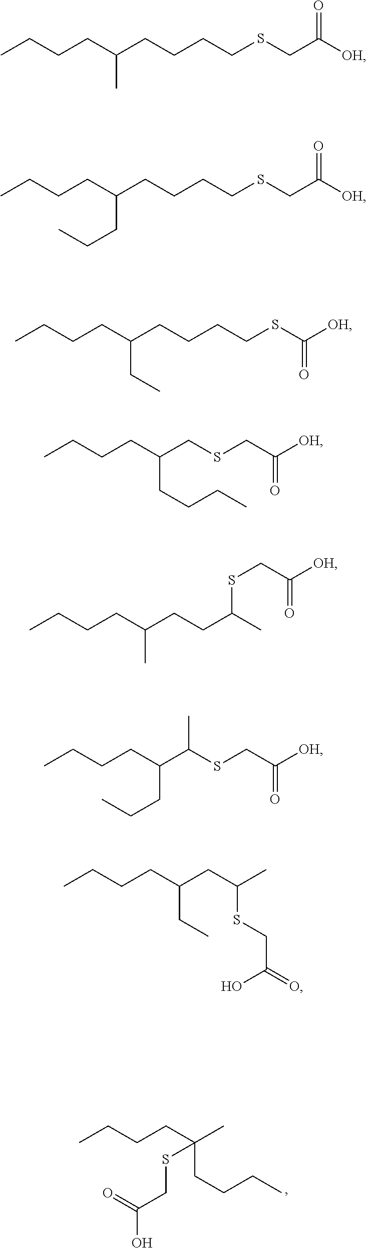

or combinations thereof.

Aspect 10. The mining chemical collector composition according to any one of the preceding Aspects, further comprising a second collector agent comprising one or more mercaptans, each mercaptan represented by formula (II):

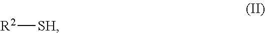

wherein R² of formula (II) is a C4-C20 hydrocarbyl.

Aspect 11. The mining chemical collector composition according to Aspect 10, wherein the one or more mercaptans represented by formula (II) comprise an n-decyl mercaptan, an n-dodecyl mercaptan (for example, Orfom® CO100 n-dodecyl mercaptan), a tert-dodecyl mercaptan (for example, a tert-dodecanethiol, such as Orfom® CO210), or combinations thereof.

Aspect 12. The mining chemical collector composition according to any one of Aspects 10-11, wherein the mining chemical collector composition comprises: a weight ratio of the one or more alkylthiocarboxylic acids represented by formula (I) to the one or more mercaptans represented by formula (II) that is in a range from about 90:10 to about 10:90, such as from about 80:20 to about 20:80, such as from about 70:30 to about 30:70, such as from about 60:40 to about 40:60, such as about 50:50 (alkylthiocarboxylic acid:mercaptan), or in a range from about 11:1 to about 1:1, such as from about 10:1 to about 2:1, such as from about 9:1 to about 3:1, such as from about 8:1 to about 4:1, such as from about 7:1 to about 5:1, such as about 6:1, or from about 0.5:1 to about 4:1, such as from about 1:1 to about 3:1, such as about 1.5:1 to about 2.5:1, such as from about 1.5:1 to about 2:1, or from about 2:1 to about 2.5:1, such as about 2:1 or about 2.5:1 (alkylthiocarboxylic acid:mercaptan).

Aspect 13. The mining chemical collector composition according to any one of the preceding Aspects, further comprising a third collector agent, the third collector agent comprising a xanthate, a xanthic ester, a xanthogen formate, a dialkyl dithiophosphate, a phosphinate, a thionocarbamate, a hydrocarbon oil, or combinations thereof.

Aspect 14. The mining chemical collector composition according to Aspect 13, wherein the third collector agent comprises the hydrocarbon oil.

Aspect 15. The mining chemical collector composition according to Aspect 14, wherein:
the hydrocarbon oil comprises a medium cycle oil;
the hydrocarbon oil comprises an aliphatic hydrocarbon, mixed aliphatic hydrocarbon, paraffinic hydrocarbon, or combinations thereof, such as a mixture of C₁₃-C₁₆ isoalkanes; or
a combination thereof.

Aspect 16. The mining chemical collector composition according to any one of Aspects 13-15, wherein a weight ratio of the one or more alkylthiocarboxylic acids represented by formula (I) to the third collector agent is in a range from about 10:1 to about 1:10, such as from about 7:1 to about 1:7, such as from about 4:1 to about 1:4, such as from about 3:1 to about 1:3, such as from about 2:1 to about 1:2, such as about 1:2 or about 2:1, or about 1.5:1 (alkylthiocarboxylic acid:third collector agent).

Aspect 17. The mining chemical collector composition according to any one of Aspects 13-16, wherein:
the mining chemical collector composition comprises one or more mercaptans represented by formula (II); and
a weight ratio of the one or more mercaptans represented by formula (II) to the third collector agent is in a range from about 5:1 to about 1:5, such as from about 4:1 to about 1:4, such as from about 3:1 to about 1:3, such as from about 2:1 to about 1:2, or from about 5:95 to about 75:25, such as from about 10:90 to about 50:50, such as from about 15:85 to about 45:55, such as from about 20:80 to about 40:60, such as from about 25:75 to about 35:65, or from 15:85 to about 25:75, such as about 20:80 (mercaptan:third collector agent).

Aspect 18. The mining chemical collector composition according to any one of the preceding Aspects, further comprising: water, an organic solvent, a pH control agent, a frothing agent, or combinations thereof.

Aspect 19. The mining chemical collector composition according to Aspect 18, wherein the water comprises tap water, distilled water, well water, osmosis water, ground water, lake water, pond water, sea water, rainwater, or combinations thereof.

Aspect 20. The mining chemical collector composition according to any one of Aspects 18-19, wherein the mining chemical collector composition comprises an amount of the water of about 75 wt % or more, 95 wt % or more, 99 wt % or more, or in a range from about 75 wt % to about 99.99 wt %, such as from about 95 wt % to about 99.99 wt % based on a total weight of the mining chemical collector composition, the total weight of the mining chemical collector composition equal to 100 wt %.

Aspect 21. The mining chemical collector composition according to any one of Aspects 18-20, wherein the frothing agent comprises any suitable frothing agent, such as pine oil; an alcohol (for example, methyl isobutyl carbinol (MIBC)); a polyether alcohol; a combination of products produced by the distillation of products from a 2-ethyl-1-hexanol manufacturing process; a mixture of heavies made during the manufacturing process of 2-ethylhexanol; or combinations thereof.

Aspect 22. The mining chemical collector composition according to any one of Aspects 18-21, wherein the pH control agent comprises any suitable pH control agent, such as lime, a carbonate compound, sulfuric acid, or combinations thereof.

Aspect 23. The mining chemical collector composition according to any one of the preceding Aspects, wherein the mining chemical collector composition has a pH in a range from about 4 to about 12, such as from about 6 to about 12, such as from about 7 to about 11, such as from about 7 to about 9, or from about 8 to about 12, such as from about 9 to about 11, such as from about 10 to about 11, such as about 10, or about 10.5, or about 11.

Aspect 24. A mining chemical collector composition to recover a compound or mineral comprising one or more metals, comprising: the mining chemical collector composition according to any one of the preceding Aspects.

Aspect 25. The mining chemical collector composition according to Aspect 24, wherein the one or more metals comprise one or more Group 3-16 metals, or one or more Group 3-14 metals, or one or more Group 3-12 metals, or one or more Group 3-11 metals, or one or more Group 5-12 metals.

Aspect 26. The mining chemical collector composition according to any one of Aspects 24-25, wherein the one or more metals comprise copper, molybdenum, iron, gold, silver, platinum, nickel, lead, zinc, cobalt, chromium, palladium, tellurium, uranium, or combinations thereof.

Aspect 27. The mining chemical collector composition according to any one of Aspects 24-26, wherein the one or more metals comprises copper, molybdenum, iron, or combinations thereof.

Aspect 28. The mining chemical collector composition according to any one of Aspects 24-27, wherein the compound or mineral comprising the one or more metals comprises a sulfide, an oxide, a mixed sulfide/oxide, or combinations thereof.

Aspect 29. The mining chemical collector composition according to any one of Aspects 24-28, wherein the compound or mineral comprising the one or more metals comprises copper sulfide, copper oxide, molybdenum sulfide, molybdenum oxide, iron sulfide, iron oxide, or combinations thereof.

Aspect 30. The mining chemical collector composition according to any one of Aspects 24-29, wherein the mining chemical collector composition preferentially extracts a copper-containing compound or mineral and a molybdenum-containing compound or mineral relative to an iron-containing compound or mineral.

Aspect 31. A copper collector composition, comprising the mining chemical collector composition according to any one of Aspects 1-30.

Aspect 32. A molybdenum collector composition, comprising: the mining chemical collector composition according to any one of Aspects 1-30.

Aspect 33. A flotation composition, comprising: the mining chemical collector composition according to any one of Aspects 1-30.

Aspect 34. A process for recovery of a metal from an ore, the process comprising: contacting an ore with a mining chemical collector composition, the mining chemical collector composition comprising a first collector agent comprising one or more alkylthiocarboxylic acids, each alkylthiocarboxylic acid represented by formula (I):

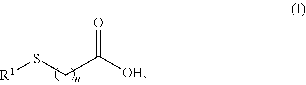

wherein: R of formula (I) is a C4-C20 hydrocarbyl, such as a C5-C18 hydrocarbyl, such as a C6-C16 hydrocarbyl; n of formula (I) is 1 or 2; and M of formula (I) is hydrogen (H), Li, Na, K, Rb, or Cs.

Aspect 35. The process according to Aspect 34, wherein the mining chemical collector composition comprises any suitable mining chemical collector composition described herein (for example, the mining chemical collector composition according to any one of Aspects 1-33).

Aspect 36. The process according to any one of Aspects 34-35, wherein the contacting the ore with the mining chemical collector composition is performed at least once during a metal flotation procedure.

Aspect 37. The process according to any one of Aspects 34-36, wherein the contacting is performed at a pH in a range from about 4 to about 12, such as from about 6 to about 12, such as from about 7 to about 11, such as from about 7 to about 9, or from about 8 to about 12, such as from about 9 to about 11, such as from about 10 to about 11, such as about 10, or about 10.5, or about 11.

Aspect 38. The process according to any one of Aspects 34-37, wherein the metal comprises copper, molybdenum, iron, gold, silver, platinum, nickel, lead, zinc, cobalt, chromium, palladium, tellurium, uranium, or combinations thereof.

Aspect 39. The process according to any one of Aspects 34-38, wherein the ore is in the form of particles during the contacting the ore with the mining chemical collector composition.

Aspect 40. The process according to any one of Aspects 34-39, wherein the ore comprises a copper-containing ore, a molybdenum-containing ore, an iron-containing ore, or combinations thereof.

Aspect 41. The process according to any one of Aspects 34-40, wherein the metal is in the form of a mineral or compound, the mineral or compound comprising a copper sulfide, a copper oxide, a molybdenum sulfide, a molybdenum oxide, an iron sulfide, an iron oxide, or combinations thereof.

Aspect 42. The process according to any one of Aspects 34-41, wherein the mining chemical collector composition preferentially extracts copper relative to iron.

Aspect 43. The process according to any one of Aspects 34-42, wherein the mining chemical collector composition preferentially extracts molybdenum relative to iron.

Aspect 44. The process according to any one of Aspects 34-43, wherein the mining chemical collector composition preferentially extracts copper and molybdenum relative to iron.

Aspect 45. The process according to any one of Aspects 34-44, wherein the mining chemical collector composition preferentially extracts copper relative to molybdenum or molybdenum relative to copper.

Aspect 46. The process according to any one of Aspects 34-45, wherein the mining chemical collector composition comprises: an amount of the one or more alkylthiocarboxylic acids represented by formula (I) that is in a range from about 3 grams per ton of the ore (g/t) to about 300 g/t, such as from about 6 g/t to about 150 g/t, such as from about 12 g/t to about 75 g/t, such as from about 24 g/t to about 37.5 g/t, such as about 30 g/t, or from about 4 to about 80 g/t, such as from about 8 g/t to about 40 g/t, such as from about 16 g/t to about 25 g/t, such as about 20 g/t.

Aspect 47. The process according to Aspect 46, wherein the mining chemical collector composition comprises:
a second collector agent comprising one or more mercaptans represented by formula (II); and
an amount of the one or more mercaptans represented by formula (II) that is in a range from about 0.5 g/t of the ore to about 50 g/t, such as from about 1 g/t to about 25 g/t, such as from about 2 g/t to about 12.5 g/t, such as from about 4 g/t to about 10 g/t, such as from about 5 g/t to about 8 g/t, such as from about 4 g/t to about 7 g/t, such as about 5 g/t.

Aspect 48. The process according to any one of Aspects 46-47, wherein the mining chemical collector composition comprises:
a third collector agent comprising a xanthate, a xanthic ester, a xanthogen formate, a dialkyl dithiophosphate, a phosphinate, a thionocarbamate, a hydrocarbon oil, or combinations thereof, and
an amount of the third collector agent that is in a range from about 1 g/t of the ore to about 100 g/t, such as from about 2 g/t to about 60 g/t, such as from about 4 g/t to about 40 g/t, such as from about 6 g/t to about 30 g/t, such as from about 8 g/t to about 25 g/t, such as from about 10 g/t to about 20 g/t, such as about 10 g/t or about 20 g/t.

Aspect 49. The process according to any one of Aspects 34-45, wherein the mining chemical collector composition comprises: an amount of the one or more alkylthiocarboxylic acids represented by formula (I) in a range from about 1 g/t of the ore to about 100 g/t, such as from about 2 g/t to about 50 g/t, such as from about 4 g/t to about 25 g/t, such as from about 8 g/t to about 12.5 g/t, such as about 10 g/t.

Aspect 50. The process according to Aspect 49, wherein the mining chemical collector composition comprises:
a second collector agent comprising one or more mercaptans represented by formula (II); and
an amount of the one or more mercaptans represented by formula (II) that is in a range from about 0.5 g/t of the ore to about 50 g/t, such as from about 1 g/t to about 25 g/t, such as from about 2 g/t to about 12.5 g/t, such as from about 4 g/t to about 10 g/t, such as from about 5 g/t to about 8 g/t, such as about 6 g/t or about 7 g/t, or from about 4 g/t to about 7 g/t, such as from about 5 g/t to about 6 g/t, such as about 5 g/t.

Aspect 51. The process according to any one of Aspects 46-47, wherein the mining chemical collector composition comprises:
a third collector agent comprising a xanthate, a xanthic ester, a xanthogen formate, a dialkyl dithiophosphate, a phosphinate, a thionocarbamate, a hydrocarbon oil, or combinations thereof, and
an amount of the third collector agent that is in a range from about 2 g/t of the ore to about 200 g/t, such as from about 4 g/t to about 100 g/t, such as from about 8 g/t to about 50 g/t, such as from about 10 g/t to about 30 g/t, such as from about 15 g/t to about 25 g/t, such as about 20 g/t.

Aspect 52. The process according to any one of Aspects 34-51, wherein:
a percent recovery of copper from the ore is about 70 wt % or more, such as about 75 wt % or more, such as about 80 wt % or more, such as about 85 wt % or more, such as about 90 wt % or more, etc., and up to about 99 wt % or less, such as up to about 95 wt % or less, etc.;
a percent recovery of copper from the ore using the mining chemical collector composition is greater (for example, by about 1 wt % or more, about 2 wt % or more, about 3 wt % or more, about 4 wt % or more, about 5 wt % or more, about 7 wt % or more, about 9 wt % or more, about 10 wt % or more, about 12 wt % or more, about 15 wt % or more, about 20 wt % or more, etc., and up to about 99 wt % or less, such as up to about 75 wt % or less, such as up to about 50 wt % or less, etc.) than a percent recovery of the composition without the one or more alkylthiocarboxylic acids represented by formula (I) under the same flotation conditions; or
a combination thereof.

Aspect 53. The process according to any one of Aspects 34-52, wherein:
a percent grade of copper recovered about 1 wt % or more, or about 2 wt % or more, or about 2.5 wt % or more, or about 3 wt % or more, about 4 wt % or more, etc., and up to about 30 wt % or less, such as up to about 10 wt % or less, etc.;
a percent grade of copper recovered using the mining chemical collector composition is greater (for example, by about 1 wt % or more, about 2 wt % or more, about 3 wt % or more, etc., and up to about 10 wt % or less, such as up to about 5 wt % or less, etc.) than that of the composition without the one or more alkylthiocarboxylic acids represented by formula (I) under the same flotation conditions; or
a combination thereof.

Aspect 54. The process according to any one of Aspects 34-53, wherein:
a percent recovery of molybdenum from the ore is about 50 wt % or more, such as about 55 wt % or more, such as about 60 wt % or more, such as about 65 wt % or more, such as about 70 wt % or more, etc., and up to about 95 wt % or less, such as up to about 90 wt % or less, etc.;
a percent recovery of molybdenum from the ore using the mining chemical collector composition is greater (for example, by about 1 wt % or more, about 2 wt % or more, about 3 wt % or more, about 4 wt % or more, about 5 wt % or more, about 7 wt % or more, about 9 wt % or more, about 10 wt % or more, about 12 wt % or more, about 15 wt % or more, about 20 wt % or more, etc., and up to about 99 wt % or less, such as about 75 wt % or less, such as up to about 50 wt % or less, etc.) than the percent recovery of the composition without the one or more alkylthiocarboxylic acids represented by formula (I) under the same flotation conditions; or
a combination thereof.

Aspect 55. The process according to any one of Aspects 34-54, wherein:
a percent grade of molybdenum recovered is about 0.05 wt % or more, about 0.08 wt % or more, about 0.1 wt % or more, or about 0.12 wt % or more, or about 0.14 wt % or more, or about 0.15 wt % or more, or about 0.16 wt % or more, etc., and up to about 3 wt % or less, such as up to about 2 wt % or less, etc.;
a percent grade of molybdenum recovered using the mining chemical collector composition is greater (for example, about 1.1×, or about 1.2×, or about 1.5× or more, or about 2× or more, etc., and up to about 3× or less, such as up to about 2.5× or less, etc.) than that of the composition without the one or more alkylthiocarboxylic acids represented by formula (I) under the same flotation conditions; or
a combination thereof.

Aspect 56. The process according to any one of Aspects 34-55, wherein:
a percent recovery of iron from the ore is 5 wt % or more, such as about 10 wt % or more, such as about 20 wt % or more, such as about 25 wt % or more, such as about 30 wt % or more, such as about 35 wt % or more, etc., and up to about 60 wt % or less, such as up to about 50 wt % or less, such as up to about 40 wt % or less, such as up to about 30 wt % or less, such as up to about 20 wt % or less, etc.;
a percent recovery of iron from the ore using the mining chemical collector composition is lower (for example, by about 1 wt % or more, about 2 wt % or more, about 3 wt % or more, about 4 wt % or more, about 5 wt % or more, about 7 wt % or more, about 9 wt % or more, about 10 wt % or more, about 12 wt % or more, about 15 wt % or more, about 20 wt % or more, etc., and up to about 50 wt % or less, such as up to about 30 wt % or less, such as up to about 10 wt % or less, etc.) than the percent recovery of the composition without the one or more alkylthiocarboxylic acids represented by formula (I) under the same flotation conditions; or
a combination thereof.

Aspect 57. The process according to any one of Aspects 34-56, wherein:
a percent grade of iron recovered is about 5 wt % or more, about 6 wt % or more, about 7 wt % or more, or about 8 wt % or more, or about 9 wt % or more, or about 10 wt % or more, or about 11 wt % or more, about 12 wt % or more, etc., and up to about 40 wt % or less, such as up to about 30 wt % or less, such as up to about 20 wt % or less, such as up to about 10 wt % or less, etc.;
a percent grade of iron recovered using the mining chemical collector composition is greater (for example, by about 1 wt % or more, about 2 wt % or more, about 3 wt % or more, about 4 wt % or more, etc., and up to about 15 wt % or less, such as up to about 10 wt % or less, such as up to about 5 wt % or less, etc.) than that of the composition without the one or more alkylthiocarboxylic acids represented by formula (I) under the same flotation conditions; or
a combination thereof.

Aspect 58. The process according to any one of Aspects 34-57, wherein: a mass recovery from the ore is greater than 0 wt %; and/or a mass recovery from the ore is about 10 wt % or less, such as about 9 wt % or less, such as about 8 wt % or less, such as about 7 wt % or less, such as about 6 wt % or less, such as about 5 wt % or less.

Aspect 59. The process according to any one of Aspects 34-56, further comprising: recovering a metal from the ore.

Aspect 60. A composition, comprising: a mining chemical collector composition described herein; and an ore.

Aspect 61. The composition according to Aspect 60, wherein the mining chemical collector composition comprises the composition according to any one of Aspects 1-33.

Aspect 62. The composition according to any one of Aspects 60-61, wherein the composition is made during performance of the process according to any one of Aspects 34-59.

Aspect 63. A mining chemical collector composition characterized as having one or more of the following flotation characteristics:
a percent recovery of copper from ore that is greater than that of the composition without the one or more alkylthiocarboxylic acids represented by formula (I) under the same flotation conditions;
a percent recovery of molybdenum from ore that is greater than that of the composition without the one or more alkylthiocarboxylic acids represented by formula (I) under the same flotation conditions;
a percent recovery of iron from ore that is less than that of the composition without the one or more alkylthiocarboxylic acids represented by formula (I) under the same flotation conditions; or
combinations thereof.

Aspect 63. The mining chemical collector composition according to Aspect 60, wherein the mining chemical collector composition comprises any mining chemical collector composition described herein (for example, the composition according to any one of Aspects 1-33).

Aspect 64. A mining chemical collector composition, comprising:
a first collector agent comprising one or more alkylthiocarboxylic acids, each alkylthiocarboxylic acid represented by formula (I):

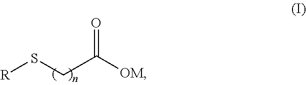

wherein: R¹ of formula (I) is a C4-C20 hydrocarbyl, n of formula (I) is 1 or 2, and M of formula (I) is hydrogen (H), Li, Na, K, Rb, or Cs; and
a second collector agent comprising a xanthate, a xanthic ester, a xanthogen formate, a dialkyl dithiophosphate, a phosphinate, a thionocarbamate, a hydrocarbon oil, one or more mercaptans, or combinations thereof, wherein each mercaptan of the one or more mercaptans is represented by formula (II):

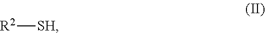

wherein: R² of formula (II) is an unsubstituted C4-C20 hydrocarbyl, the unsubstituted C4-C20 hydrocarbyl being linear or branched, cyclic or acyclic.

Aspect 65. A mining chemical collector composition, comprising:
first collector agent comprising one or more alkylthiocarboxylic acids, each alkylthiocarboxylic acid represented by formula (I):

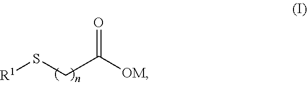

wherein: R¹ of formula (I) is a C4-C20 hydrocarbyl, n of formula (I) is 1 or 2, and M of formula (I) hydrogen (H), Li, Na, K, Rb, or Cs; and
a second collector agent comprising one or more mercaptans, each mercaptan represented by formula (II):

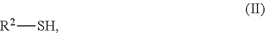

wherein R² of formula (II) is a C4-C20 hydrocarbyl,
the mining chemical collector composition is characterized as having one or more of the following flotation characteristics: a percent recovery of copper from ore that is greater than that of the composition without the one or more alkylthiocarboxylic acids represented by formula (I) under the same flotation conditions; a percent recovery of molybdenum from ore that is greater than that of the composition without the one or more alkylthiocarboxylic acids represented by formula (I) under the same flotation conditions; a percent recovery of iron from ore that is less than that of the composition without the one or more alkylthiocarboxylic acids represented by formula (I) under the same flotation conditions; or combinations thereof.

Aspect 66. A process for recovery of a metal from an ore, the process comprising contacting an ore with the mining chemical collector composition according to Aspects 64-65; and optionally recovering a metal from the ore.

Aspect 67. The process according to Aspect 66, wherein the contacting the ore with the mining chemical collector composition is performed at least once during a metal flotation procedure.

Aspect 68. The process according to any one of Aspects 66-67, wherein the metal comprises one or more Group 3-16 metals.

Aspect 69. The process according to Aspect 68, wherein:
the one or more Group 3-16 metals comprises copper, molybdenum, iron, gold, silver, platinum, nickel, lead, zinc, cobalt, chromium, palladium, tellurium, uranium, or combinations thereof, and
the one or more Group 3-16 metals is in the form of a metal sulfide, metal oxide, or combinations thereof.

In the foregoing, reference is made to aspects of the disclosure. However, it should be understood that the disclosure is not limited to specific described aspects. Instead, any combination of the following features and elements, whether related to different aspects or not, is contemplated to implement and practice the disclosure. Furthermore, although aspects of the disclosure can achieve advantages over other possible solutions and/or over the prior art, whether or not a particular advantage is achieved by a given aspect is not limiting of the disclosure. Thus, the foregoing aspects, features, embodiments, implementations, and advantages are merely illustrative and are not considered elements or limitations of the appended claims except where explicitly recited in a claim(s). Likewise, reference to “the disclosure” shall not be construed as a generalization of any inventive subject matter disclosed herein and shall not be considered to be an element or limitation of the appended claims except where explicitly recited in a claim(s).

As is apparent from the foregoing general description and the specific aspects, while forms of the aspects have been illustrated and described, various modifications can be made without departing from the spirit and scope of the present disclosure. Accordingly, it is not intended that the present disclosure be limited thereby. Likewise, the term “comprising” is considered synonymous with the term “including.” Likewise whenever a formulation, a composition, an element or a group of elements is preceded with the transitional phrase “comprising,” it is understood that we also contemplate the same formulation, composition or group of elements with transitional phrases “consisting essentially of,” “consisting of,” “selected from the group of consisting of,” or “is” preceding the recitation of the formulation, composition, element, or elements and vice versa, for example, the terms “comprising,” “consisting essentially of,” “consisting of” also include the product of the combinations of elements listed after the term.

References cited herein are incorporated by reference herein in their entirety to indicate the state of the art as of their publication or filing date and it is intended that this information can be employed herein, if desired, to exclude specific aspects that are in the prior art.

For purposes of this present disclosure, and unless otherwise specified, all numerical values within the detailed description and the claims herein are modified by “about” or “approximately” the indicated value, and consider experimental error and variations that would be expected by a person having ordinary skill in the art. It will be further understood that there are a number of values disclosed therein, and that each value is also herein disclosed as “about” that particular value in addition to the value itself. In aspects, use of the term “about” can refer to ±20% of the stated value, ±15% of the stated value, ±10% of the stated value, ±5% of the stated value, ±3% of the stated value, ±2% of the stated value, or ±1% of the stated value.

For the sake of brevity, only certain ranges are explicitly disclosed herein. However, ranges from any lower limit can be combined with any upper limit to recite a range not explicitly recited, as well as, ranges from any lower limit can be combined with any other lower limit to recite a range not explicitly recited, in the same way, ranges from any upper limit can be combined with any other upper limit to recite a range not explicitly recited. Additionally, within a range includes every point or individual value between its end points even though not explicitly recited. Thus, every point or individual value can serve as its own lower or upper limit combined with any other point or individual value or any other lower or upper limit, to recite a range not explicitly recited. For example, by disclosing a wt % from 70 wt % to 80 wt %, an intent is to recite individually 70 wt %, 71 wt %, 72 wt %, 73 wt %, 74 wt %, 75 wt %, 76 wt %, 77 wt %, 78 wt %, 79 wt %, and 80 wt %, including any sub-ranges and combinations of sub-ranges encompassed therein such that any of the foregoing numbers can be used singly to describe an open-ended range or in combination to describe a close-ended range.

Moreover, all numerical end points of ranges disclosed herein are approximate, unless excluded by proviso. As a representative example, if a wt % is from 10 wt % to 75 wt %, this range should be interpreted as encompassing a wt % in a range from “about” 10 wt % to “about” 75 wt %.

As used herein, the indefinite article “a” or “an” shall mean “at least one” unless specified to the contrary or the context clearly indicates otherwise. For example, aspects comprising “an alkylthiocarboxylic acid” include aspects comprising one, two, or more alkylthiocarboxylic acids, unless specified to the contrary or the context clearly indicates only one alkylthiocarboxylic acid is included.

When a compound is described herein such that a particular isomer, enantiomer, or diastereomer of the compound is not specified, for example, in a formula or in a chemical name, that description is intended to include each isomer and enantiomer of the compound described individually or in any combination. For example, any general structure, formula, or name presented is also intended to encompass all structural isomers, conformational isomers, regioisomers, stereoisomers (such as enantiomers, diastereomers, and other optical isomers whether in enantiomeric or racemic forms, as well as mixtures of stereoisomers, as the context permits or requires) that can arise from a particular set of substituents, unless indicated otherwise. Thus, a general reference to a compound includes all structural isomers unless specified to the contrary or the context clearly indicates otherwise. For example, reference to a hydrocarbon without specifying a particular isomer (such as butyl) expressly discloses all isomers (such as n-butyl, iso-butyl, sec-butyl, and tert-butyl). For example, reference to a C5 hydrocarbon expressly discloses all isomers thereof.

While the foregoing is directed to aspects of the present disclosure, other and further aspects of the disclosure can be devised without departing from the basic scope thereof, and the scope thereof is determined by the claims that follow.

What is claimed is:

1. A mining chemical collector composition, comprising:
a first collector agent comprising one or more alkylthiocarboxylic acids, each alkylthiocarboxylic acid represented by formula (I): 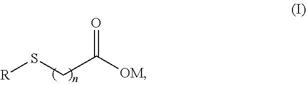
wherein:
R¹ of formula (I) is a C4-C20 hydrocarbyl;
n of formula (I) is 1 or 2; and
M of formula (I) is hydrogen (H), Li, Na, K, Rb, or Cs.

2. The mining chemical collector composition according to claim 1, wherein R¹ of formula (I) is a linear or branched, cyclic or acyclic C4-C20 hydrocarbyl.

3. The mining chemical collector composition according to claim 1, wherein R¹ of formula (I) is an unsubstituted C4-C20 hydrocarbyl.

4. The mining chemical collector composition according to claim 1, wherein R¹ of formula (I) is an unsubstituted C6-C16 hydrocarbyl.

5. The mining chemical collector composition according to claim 1, wherein the one or more alkylthiocarboxylic acids represented by formula (I) comprise an alkylthiocarboxylic acid represented by formula (I-A), formula (I-B), formula (I-C), formula (I-D), formula (I-E), formula (I-F), formula (I-G), formula (I-H), or combinations thereof: 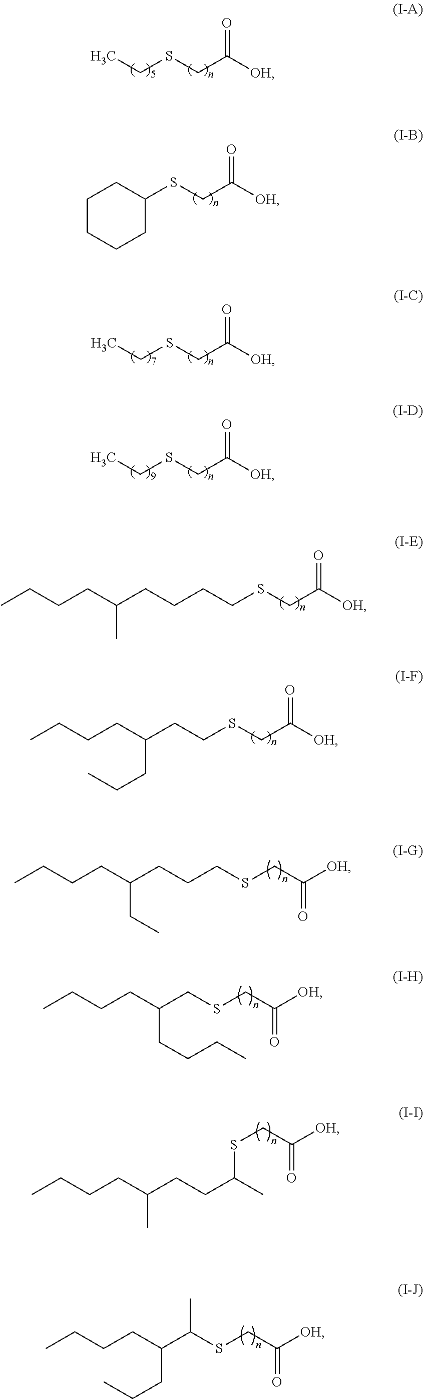 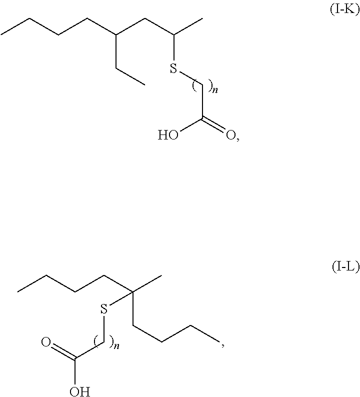
wherein n of formulas (I-A), (I-B), (I-C), (I-D), (I-E), (I-F), (I-G), (I-H), (I-I), (I-J), (I-K), and (I-L) is, independently, 1 or 2.

6. The mining chemical collector composition according to claim 1, wherein the one or more alkylthiocarboxylic acids represented by formula (I) comprise: 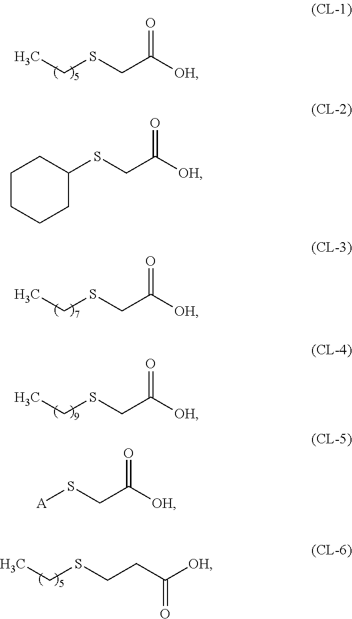
combinations thereof,
wherein A of structure CL-5 is a branched C10 hydrocarbyl.

7. The mining chemical collector composition according to claim 6, wherein:
the one or more alkylthiocarboxylic acids represented by formula (I) comprise structure CL-5; and
CL-5 comprises: 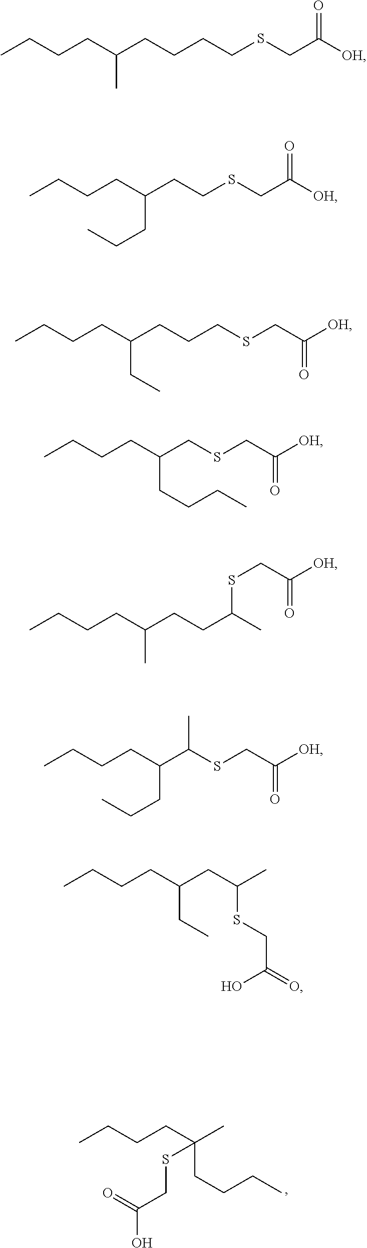
or combinations thereof.

8. The mining chemical collector composition according to claim 1, further comprising a second collector agent comprising one or more mercaptans, each mercaptan represented by formula (II): 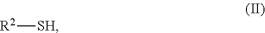
wherein R² of formula (II) is an unsubstituted C4-C20 hydrocarbyl, the unsubstituted C4-C20 hydrocarbyl being linear or branched, cyclic or acyclic.

9. The mining chemical collector composition according to claim 8, wherein the one or more mercaptans represented by formula (II) comprises n-decyl mercaptan, n-dodecyl mercaptan, tert-dodecyl mercaptan, or combinations thereof.

10. The mining chemical collector composition according to claim 8, wherein the mining chemical collector composition comprises a weight ratio of the one or more alkylthiocarboxylic acids represented by formula (I) to the one or more mercaptans represented by formula (II) that is in a range from about 11:1 to about 1:1.

11. The mining chemical collector composition according to claim 8, further comprising a third collector agent comprising a hydrocarbon oil.

12. The mining chemical collector composition according to claim 1, further comprising a third collector agent, the third collector agent comprising a xanthate, a xanthic ester, a xanthogen formate, a dialkyl dithiophosphate, a phosphinate, a thionocarbamate, a hydrocarbon oil, or combinations thereof.

13. The mining chemical collector composition according to claim 12, wherein a weight ratio of the one or more alkylthiocarboxylic acids represented by formula (I) to the third collector agent is in a range from about 10:1 to about 1:10.

14. The mining chemical collector composition according to claim 1, further comprising water, an organic solvent, a pH control agent, a frothing agent, or combinations thereof.

15. A process for recovery of a metal from an ore, the process comprising:
contacting an ore with a mining chemical collector composition, the mining chemical collector composition comprising a first collector agent comprising one or more alkylthiocarboxylic acids, each alkylthiocarboxylic acid represented by formula (I): 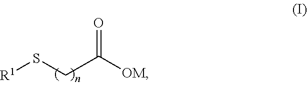
wherein:
R¹ of formula (I) is a C4-C20 hydrocarbyl;
n of formula (I) is 1 or 2; and
M of formula (I) is hydrogen (H), Li, Na, K, Rb, or Cs.

16. The process according to claim 15, wherein the contacting the ore with the mining chemical collector composition is performed at least once during a metal flotation procedure.

17. The process according to claim 15, wherein the metal comprises one or more Group 3-16 metals.

18. The process according to claim 17, wherein:
the one or more Group 3-16 metals comprise copper, molybdenum, iron, gold, silver, platinum, nickel, lead, zinc, cobalt, chromium, palladium, tellurium, uranium, or combinations thereof, and
the one or more Group 3-16 metals are in the form of a metal sulfide, metal oxide, or combinations thereof.

19. The process according to claim 15, wherein the mining chemical collector composition further comprises:
a second collector agent comprising one or more mercaptans, each mercaptan represented by formula (II): 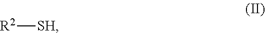
wherein: R² of formula (II) is a C4-C20 hydrocarbyl;
a third collector agent comprising a xanthate, a xanthic ester, a xanthogen formate, a dialkyl dithiophosphate, a phosphinate, a thionocarbamate, a hydrocarbon oil, or combinations thereof, or
a combination thereof.

20. A mining chemical collector composition, comprising:
first collector agent comprising one or more alkylthiocarboxylic acids, each alkylthiocarboxylic acid represented by formula (I): 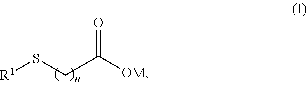
wherein: R¹ of formula (I) is a C4-C20 hydrocarbyl, n of formula (I) is 1 or 2, and M of formula (I) hydrogen (H), Li, Na, K, Rb, or Cs; and
a second collector agent comprising one or more mercaptans, each mercaptan represented by formula (II): 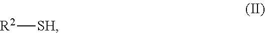
wherein R² of formula (II) is a C4-C20 hydrocarbyl,
the mining chemical collector composition is characterized as having one or more of the following flotation characteristics:
a percent recovery of copper from ore that is greater than that of the composition without the one or more alkylthiocarboxylic acids represented by formula (I) under the same flotation conditions;
a percent recovery of molybdenum from ore that is greater than that of the composition without the one or more alkylthiocarboxylic acids represented by formula (I) under the same flotation conditions;
a percent recovery of iron from ore that is less than that of the composition without the one or more alkylthiocarboxylic acids represented by formula (I) under the same flotation conditions; or
combinations thereof.

21. A process for recovery of a metal from an ore, the process comprising:
contacting an ore with a mining chemical collector composition, the mining chemical collector composition comprising a first collector agent comprising one or more alkylthiocarboxylic acids and a second collector agent comprising one or more mercaptans, each alkylthiocarboxylic acid represented by formula (I) and each mercaptan represented by formula (II): 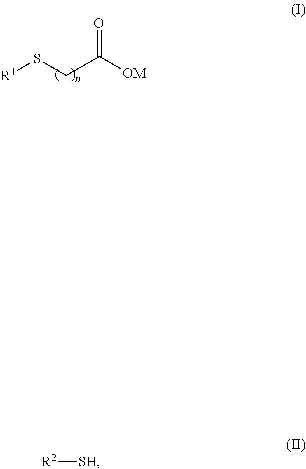
wherein:
R¹ of formula (I) is a C4-C20 hydrocarbyl, n of formula (I) is 1 or 2, and M of formula (I) hydrogen (H), Li, Na, K, Rb, or Cs; and
R² of formula (II) is a C4-C20 hydrocarbyl;
the mining chemical collector composition is characterized as having one or more of the following flotation characteristics:
a percent recovery of copper from ore that is greater than that of the composition without the one or more alkylthiocarboxylic acids represented by formula (I) under the same flotation conditions;
a percent recovery of molybdenum from ore that is greater than that of the composition without the one or more alkylthiocarboxylic acids represented by formula (I) under the same flotation conditions;
a percent recovery of iron from ore that is less than that of the composition without the one or more alkylthiocarboxylic acids represented by formula (I) under the same flotation conditions; or
combinations thereof.

22. The process according to claim 21, wherein the contacting the ore with the mining chemical collector composition is performed at least once during a metal flotation procedure.

23. The process according to claim 21, wherein the metal comprises one or more Group 3-16 metals.

24. The process according to claim 23, wherein:
the one or more Group 3-16 metals comprise copper, molybdenum, iron, gold, silver, platinum, nickel, lead, zinc, cobalt, chromium, palladium, tellurium, uranium, or combinations thereof, and
the one or more Group 3-16 metals are in the form of a metal sulfide, metal oxide, or combinations thereof.

25. A mining chemical collector composition, comprising:
a first collector agent comprising one or more alkylthiocarboxylic acids, each alkylthiocarboxylic acid represented by formula (I): 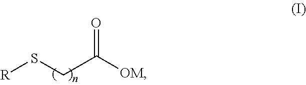
wherein: R¹ of formula (I) is a C4-C20 hydrocarbyl, n of formula (I) is 1 or 2, and M of formula (I) is hydrogen (H), Li, Na, K, Rb, or Cs; and
a second collector agent comprising a xanthate, a xanthic ester, a xanthogen formate, a dialkyl dithiophosphate, a phosphinate, a thionocarbamate, a hydrocarbon oil, one or more mercaptans, or combinations thereof, wherein each mercaptan of the one or more mercaptans is represented by formula (II): 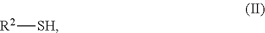
wherein: R² of formula (II) is an unsubstituted C4-C20 hydrocarbyl, the unsubstituted C4-C20 hydrocarbyl being linear or branched, cyclic or acyclic.

26. A process for recovery of a metal from an ore, the process comprising:
contacting an ore with a mining chemical collector composition comprising:
a first collector agent comprising one or more alkylthiocarboxylic acids, each alkylthiocarboxylic acid represented by formula (I): 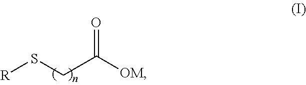
wherein: R¹ of formula (I) is a C4-C20 hydrocarbyl, n of formula (I) is 1 or 2, and M of formula (I) is hydrogen (H), Li, Na, K, Rb, or Cs; and
a second collector agent comprising a xanthate, a xanthic ester, a xanthogen formate, a dialkyl dithiophosphate, a phosphinate, a thionocarbamate, a hydrocarbon oil, one or more mercaptans, or combinations thereof, wherein each mercaptan of the one or more mercaptans is represented by formula (II): 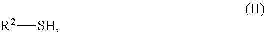
wherein: R² of formula (II) is an unsubstituted C4-C20 hydrocarbyl, the unsubstituted C4-C20 hydrocarbyl being linear or branched, cyclic or acyclic.

27. The process according to claim 26, wherein the contacting the ore with the mining chemical collector composition is performed at least once during a metal flotation procedure.

28. The process according to claim 26, wherein the metal comprises one or more Group 3-16 metals.

29. The process according to claim 28, wherein:
the one or more Group 3-16 metals comprise copper, molybdenum, iron, gold, silver, platinum, nickel, lead, zinc, cobalt, chromium, palladium, tellurium, uranium, or combinations thereof; and
the one or more Group 3-16 metals are in the form of a metal sulfide, metal oxide, or combinations thereof.
